(19) United States
(12) Patent Application Publication
(10) Pub. No.: US 2026/0123879 A1
(43) Pub. Date: May 7, 2026

(54) SYSTEMS, DEVICES, AND METHODS FOR DETEREMINING A FETAL OXIMETRY VALUE USING AN IN VIVO FETAL OXIMETRY MODEL AND/OR AN AUGMENTED IN VIVO FETAL OXIMETRY MODEL

(71) Applicant: Raydiant Oximetry, Inc., San Ramon, CA (US)

(72) Inventors: Paul Stetson, Piedmont, CA (US); Neil Padharia Ray, Sacramento, CA (US); Adam Jacobs, Hollis, NH (US); Andrew Prescott, Bedford, NH (US)

(21) Appl. No.: 19/117,389

(22) PCT Filed: Oct. 11, 2023
(86) PCT No.: PCT/US2023/034961

§ 371 Date: Mar. 31, 2025

Related U.S. Application Data
(60) Provisional application No. 63/415,269, filed on Oct. 11, 2022.

(51) Int. Cl. A61B 5/00; A61B 5/024; A61B 5/1455; A61B 5/1464

(52) U.S. Cl. CPC A61B 5/4362; A61B 5/02411; A61B 5/14551; A61B 5/1464; A61B 5/7267; A61B 5/7282; A61B 5/742; A61B 2503/02

(57) ABSTRACT

A physiological indicator for a pregnant mammal and/or her fetus may be used to augment and/or personalize a fetal oximetry model to the pregnant mammal, the fetus, and/or the pregnant mammal/fetus pair. Light transmission data that corresponds to and/or represents a composite of light that passes through the pregnant mammal's abdomen and the fetus may be received and input into the augmented fetal in vivo fetal oximetry model. An output from the augmented fetal in vivo fetal oximetry model that includes an indication of an oximetry value and/or wellness for the fetus may be generated and/or received.

### RELATED APPLICATION

This application is an INTERNATIONAL application (PCT) claiming priority to U.S. Provisional Patent Application No. 63/415,269 filed on 11 Oct. 2022 and entitled “SYSTEMS, DEVICES, AND METHODS FOR DETERMINING AFETAL OXIMETRY VALUE USING AN IN VIVO FETAL OXIMETRY MODEL AND/OR AN AUGMENTED IN VIVO FETAL OXIMETRY MODEL,” which is incorporated by reference, in its entirety, herein.

### FIELD OF INVENTION

The present invention is in the field of medical devices, oximetry, pulse oximetry, and machine learning, more particularly, in the fields using machine learning to develop a model to determine and/or predict fetal oximetry values using measured light transmission data, wherein a portion of the measured light transmission data includes light transmission data for 301 light incident on a fetus within a pregnant mammal's abdomen. The present invention is also directed to using a fetal oximetry model to determine and/or predict fetal oximetry values using measured light transmission data.

### BACKGROUND

Current methods of monitoring fetal health, such as monitoring fetal heart rate, are inefficient and prone to inaccuracies when determining levels of fetal distress and, at times, provide false positive results indicating fetal distress that may result in the unnecessary performance of a Cesarean delivery. One area of interest in improving fetal health monitoring includes the use of transabdominal fetal oximetry.

Oximetry is a method for determining a level of oxygen saturation of a mammal's tissue, arterial hemoglobin, and/or venous hemoglobin. A mammal's level of oxygen saturation may provide an indication of health or overall wellness of an individual. Transabdominal-fetal-oximetry is a method of oximetry for a fetus performed by analyzing light projected into a pregnant mammal's abdomen that reflects off the fetus contained therein and is detected by a photodetector. The optical information detected by the photodetector is analyzed to calculate fetal oximetry values that may be used to determine whether or not a fetus is in distress and/or is at risk of developing hypoxemia or hypoxia.

### SUMMARY

Systems, devices, and methods disclosed herein may receive a physiological indicator such as a hemoglobin oxygen saturation level, a tissue oxygen saturation level, a skin tone, a heartrate, a blood pressure, photoplethysmogram (PPG) signals or values, pulse, respiratory rate, uterine contraction information, duration of labor and delivery of a fetus, gestational age of the fetus, chronological age of the pregnant mammal, a hemoglobin oxygen saturation level, and a tissue oxygenation level for a pregnant mammal or the pregnant mammal's fetus.

Light transmission data that corresponds to an optical signal that is detected by a photodetector and converted into the light transmission data may be received. The optical signal may be a composite of light that passes through the pregnant mammal's abdomen and a fetus disposed within the pregnant mammal's abdomen.

The light transmission data may be input into an augmented fetal in vivo fetal oximetry model that incorporates the physiological indicator as, for example, in input variable of an in vivo fetal oximetry model. In some embodiments, personalization of the fetal oximetry model may be done by determining a calibration formula for the pregnant mammal or fetus responsively to the physiological indicator and then incorporating the determined calibration formula into a fetal oximetry model. At times, determining the calibration formula may include querying a database of calibration equations for a calibration equation that matches and/or is responsive to the physiological indicator. A calibration equation may be received from the database responsively to the query and the augmented fetal in vivo fetal oximetry model may be personalized to the pregnant mammal or fetus using the received calibration equation. Additionally, or alternatively, personalization of the fetal oximetry model may be done by selecting and/or determining a physiological indicator model for the pregnant mammal or fetus responsively to the physiological indicator and then incorporating the selected and/or determined physiological indicator model into a fetal oximetry model. At times, selecting and/or determining the physiological indicator model may include querying a database of calibration equations for a physiological indicator model that matches and/or is responsive to the physiological indicator. A physiological indicator model may be received from the database responsively to the query and the augmented fetal in vivo fetal oximetry model may be personalized to the pregnant mammal or fetus using the received physiological indicator model.

An output from the augmented fetal in vivo fetal oximetry model may be received. The output may include an indication of an oximetry value for the fetus such as a level of fetal hemoglobin oxygen saturation and/or a level of fetal tissue oxygen saturation. In some embodiments, an indication of fetal distress may be determined using the oximetry value for the fetus and the indication of fetal distress may be provided to a display device. In some embodiments, the oximetry value for the fetus may be compared to a threshold fetal oximetry value to, for example, determine whether or not the fetus may be in distress. An indication of the comparison may then be compared to a display device.

In some embodiments, a plurality of physiological indicators may be received over time. The plurality of physiological indicators may be evaluated, and the augmented fetal in vivo fetal oximetry model may be personalized to the pregnant mammal or fetus using an evaluation of the received plurality of physiological indicators. At times, the plurality of physiological indicators may be a series of measurements (e.g., fetal heart rate or fetal ECG measurements) received over time and the evaluation of the plurality of physiological indicators may be a trend analysis and/or a time-weighted average analysis.

Additionally, or alternatively, systems, devices, and methods disclosed herein may determine information regarding a blood oxygen value of a fetus by obtaining at least one signal indicating light detected from a pregnant mammal's abdomen and/or a fetus disposed in the pregnant mammal's abdomen following application of light to the pregnant mammal's abdomen. The at least one signal may then be analyzed using a trained model personalized to the pregnant mammal or fetus to determine blood oxygen information used to determine the information regarding a blood oxygen value (e.g., fetal hemoglobin oxygen saturation and/or fetal tissue oxygen saturation) of the fetus. At times, the fetal blood oxygen value may be used to determine whether the fetus has fetal hypoxia or fetal hypoxemia, and an indication of this determination may be provided to a display device.

The trained model may be augmented and/or personalized to the pregnant mammal or fetus using a physiological indicator for the pregnant mammal or fetus. Additionally, or alternatively, the trained model may be personalized to the pregnant mammal or fetus using at least one of an optical and a geometric characteristic of the pregnant mammal's abdomen or fetus. Additionally, or alternatively, the trained model may be personalized to the pregnant mammal or fetus using a characteristic of the at least one signal. Additionally, or alternatively, the trained model may be personalized to the pregnant mammal and/or fetus using a light scattering coefficient and/or a light absorption coefficient specific to the pregnant mammal or fetus. Additionally, or alternatively, the trained model may be personalized to the pregnant mammal and/or fetus using a skin color of the pregnant mammal's abdomen and/or the fetus, an analysis of an image of the pregnant mammal's abdomen, and/or a calibration formula that is responsive to the physiological indicator.

Additionally, or alternatively, systems, devices, and methods disclosed herein may determine information regarding a blood oxygen value of a patient, such as a fetus, pregnant mammal, and/or non-pregnant adult human being by obtaining a physiological indicator for the patient and at least one signal indicating light detected from the patient following application of light to the patient and analyzing the at least one signal using at least one trained model and the physiological indicator of the patient to determine blood oxygen information (e.g., hemoglobin oxygen saturation and/or tissue oxygen saturation levels) for the patient. At times, the blood oxygen value may be used to determine whether the patient has hypoxia or hypoxemia using the blood oxygen value and an indication of this determination may be provided to a display device. The trained model may be personalized to the patient using the physiological indicator, a skin tone of the patient, skin melanin content for the patient, a characteristic of the at least one signal, a light scattering coefficient specific to the patient, and/or a light absorption coefficient specific to the patient.

Additionally, or alternatively, systems, devices, and methods disclosed herein may receive optical data regarding the pregnant mammal's abdomen and physiological data for the pregnant mammal and generating a physiological indication model for the pregnant mammal responsively to the received optical data and the received physiological data. On some occasions, light transmission data may be received. The light transmission data may correspond to an optical signal that is incident on the pregnant mammal's abdomen and the fetus disposed within the pregnant mammal's abdomen and is detected by a photodetector and converted into the light transmission data. An oximetry value for the fetus may be determined by applying the physiological indicator model to the light transmission data. The light transmission data may be received from, for example, a frequency domain near infrared spectroscopy system, a continuous wave near infrared spectroscopy system, and a time of flight near infrared spectroscopy system.

In some embodiments, the optical signal may be a first optical signal and the photodetector may be a first photodetector of a first spectroscopy system and the method may further comprise receiving a second set of light transmission data corresponding a second optical signal that is incident on the pregnant mammal's abdomen and a fetus disposed within the pregnant mammal's abdomen and is detected by a photodetector of a second spectroscopy system and converted into the second set of light transmission data and an oximetry value for the fetus may be determined by applying the calibration equation to the second set of light transmission data. At times, the physiological data for the pregnant mammal may include an oxygen saturation level for the pregnant mammal that may be received from a pulse oximeter.

### BRIEF DESCRIPTION OF THE FIGURES

The present invention is illustrated by way of example, and not limitation, in the figures of the accompanying drawings in which:

FIG. 1A is a block diagram illustrating an exemplary system for developing a model to accurately calculate fetal oxygen saturation in-utero, consistent with some embodiments of the present invention;

FIG. 1B is a block diagram of an exemplary system for detecting and/or determining fetal hemoglobin oxygen saturation levels, consistent with some embodiments of the present invention;

FIG. 1C is a block diagram of an exemplary fetal oximetry probe that may be used in the system of FIG. 1B, consistent with some embodiments of the present invention;

FIG. 2A is a flowchart showing an exemplary process for generating a plurality of sets of simulated light transmission data and corresponding oximetry values using a computer-generated model of animal tissue, consistent with some embodiments of the present invention;

FIG. 2B depicts a graph plotting exemplary relationships between a ratio of ratios (R) and a hemoglobin oxygen saturation percentage of a fetus for various fetal depths, consistent with some embodiments of the present invention;

FIG. 2C depicts a graph that plots exemplary relationships between a ratio of a change in an absorption coefficient for an infrared wavelength of light divided by a ratio of a change in an absorption coefficient for a red wavelength of light is related to a hemoglobin oxygen saturation percentage of a fetus for various fetal depths, consistent with some embodiments of the present invention;

FIG. 3A is a flowchart showing an exemplary process for generating a plurality of sets of simulated light transmission data and corresponding oximetry values using light transmitted through a physical model of animal tissue, consistent with some embodiments of the present invention;

FIG. 3B is a flowchart illustrating an exemplary process for developing and/or training a physiological indicator model, in accordance with some embodiments of the present invention;

FIG. 4A is a flowchart illustrating a first part of an exemplary process for developing a model to compensate for the physio-optical influences of transabdominal fetal oximetry in order to accurately calculate fetal oxygen saturation in-utero, in accordance with some embodiments of the present invention;

FIG. 4B is a flowchart illustrating a second part of the exemplary process of FIG. 4A, in accordance with some embodiments of the present invention;

FIG. 5 is a flowchart illustrating a process for the generation of a tuned simulated fetal oximetry model and/or simulated augmented fetal oximetry model, consistent with some embodiments of the present invention;

FIG. 6 is a flowchart illustrating a process for the generation of a tuned in vivo fetal oximetry model and/or augmented in vivo fetal oximetry model, consistent with some embodiments of the present invention;

FIG. 7 is a flowchart illustrating an exemplary process for the generation of an in vivo fetal oximetry model and/or augmented in vivo fetal oximetry model, consistent with some embodiments of the present invention;

FIG. 8 is a flowchart illustrating a process for the determination of an oximetry value for a fetus using an in vivo fetal oximetry model and/or augmented in vivo fetal oximetry model, consistent with some embodiments of the present invention;

FIG. 9 is a diagram showing an exemplary seven-layer two-dimensional model of a pregnant mammal's abdomen and fetus is shown, in accordance with some embodiments of the present invention;

FIG. 10 is a table of an exemplary set of parameters, in accordance with some embodiments of the present invention;

FIG. 11 provides a graph that plots a simulated fetal and maternal photoplethysmogram (PPG) over time in seconds, in accordance with some embodiments of the present invention;

FIG. 12 provides a flowchart of an exemplary process for using an in vivo fetal oximetry model and/or augmented in vivo fetal oximetry model to determine a fetal oxygenation value, in accordance with some embodiments of the present invention;

FIG. 13 provides a flowchart of an exemplary process for selecting a calibration formula for use with an in vivo fetal oximetry model and/or augmented in vivo fetal oximetry model and determine a fetal oxygenation value using the calibration formula and an in vivo fetal oximetry model and/or augmented in vivo fetal oximetry model, in accordance with some embodiments of the present invention;

FIG. 14 provides a flowchart of an exemplary process for determining optical properties of maternal tissue, selecting a calibration formula for use with an in vivo fetal oximetry model and/or augmented in vivo fetal oximetry model responsively to the maternal optical properties, and determining a fetal oxygenation value using the calibration formula and an in vivo fetal oximetry model and/or augmented in vivo fetal oximetry model, in accordance with some embodiments of the present invention;

FIG. 15 provides a flowchart of another exemplary process for determining optical properties of maternal tissue, selecting a calibration formula for use with an in vivo fetal oximetry model and/or augmented in vivo fetal oximetry model responsively to the maternal optical properties, and determining a fetal oxygenation value using the calibration formula and an in vivo fetal oximetry model and/or augmented in vivo fetal oximetry model, in accordance with some embodiments of the present invention;

FIG. 16 provides a flowchart of an exemplary process for using a fetal oximetry model and/or augmented fetal in vivo fetal oximetry model to determine a fetal oxygenation value, in accordance with some embodiments of the present invention;

FIG. 17 provides a flowchart of an exemplary process for determining a fetal oximetry value of using a calibration formula and an in vivo fetal oximetry model and/or augmented in vivo fetal oximetry model, in accordance with some embodiments of the present invention;

FIG. 18 provides a flowchart of an exemplary process for generating a fetal signal, in accordance with some embodiments of the present invention; and

FIG. 19 provides a flowchart of an exemplary process for generating a fetal signal, in accordance with some embodiments of the present invention;

FIG. 20 provides a flowchart of an exemplary process for determining an indication of an oximetry value for a fetus, in accordance with some embodiments of the present invention; and

FIG. 21 is a screen shot of an exemplary user interface configured to display an oximetry value and/or indication of fetal distress, in accordance with some embodiments of the present invention.

Throughout the drawings, the same reference numerals, and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

Near-IR spectroscopy, and pulse oximetry calculations, to estimate a percentage of oxygen bound to hemoglobin in the blood (also referred to herein as “hemoglobin oxygen saturation” or “SpO2”) may incorporate calculations using the modified Beer Lambert law (mBLL), which describes changes in hemoglobin absorption related to changes in light intensity of various wavelengths. Under the assumption that bulk tissue has homogenous characteristics, the modified Beer Lambert law can be written as Equation 1:

$${Δμ}_{a} ( λ ) = - \frac{1}{r * DPF ( λ )} \frac{{I}_{d ( λ )} - {I}_{s ( λ )}}{{I}_{s ( λ )}} = - \frac{1}{r * DPF ( λ )} Δ OD \qquad Equation 1$$

Where:
   I_{d}=diastolic intensity of the fetal pulse
   Iₛ=systolic intensity of the fetal pulse
   r=source detector distance, which may be given by the geometry of an optical probe or source/detector combination
   DPF=differential path length factor, which is not known
   ΔOD=change in optical density
   Δμₐ=change in absorption coefficient
   λ=wavelength

Results from this equation may be used to extract values for concentrations of oxygenated hemoglobin (sometimes referred to herein as “c_HbO”) and deoxygenated hemoglobin (sometimes referred to herein as “c_Hb”) 2 according to Equation 2, below.

$$Sp O 2 ( \% ) = c\_HbO / ( c\_HbO + c\_Hb ) \qquad Equation 2$$

Although the modified Beer Lambert Law traditionally serves as the fundamental basis for near-IR spectroscopy, it is limited by several assumptions, including that light absorption within tissue is homogeneous, change in a differential path length factor is negligible, and that the light scattering within tissue is low. In complex in vivo, physio-optical environments of, for example, non-homogenous tissue and/or two or more layers of different types of tissue such as a fetus within a mother, these assumptions may not always hold true and yield accurate calculations. For example, traditional methods for calculating pulse oximetry using the modified Beer Lambert Law assume that photons travel a relatively short distance (e.g., 1 cm) and that there is a negligible change in the differential path length factor (DPF) across this distance. However, when a distance photons travel is larger than 1 cm (e.g., 1.5-10 cm) changes in the DPF can be significant thereby adversely impacting the accuracy SpO2 calculations. For example, in transabdominal fetal application, photons can travel 5 cm, 10 cm, or more and, consequently, changes in the DPF can be significant. To accurately account for DPF variability and calculate oxygen saturation is a complex problem, particularly when multiple wavelengths of light are used. One way to overcome this problem is to calculate SpO2 using a 2-layer description of the modified Beer Lambert Law that can independently calibrate for different layers of tissue.

However, a drawback of the using the two-layer Modified Beer Lambert Law calculations when calculating oxygen saturation of target tissue is that this approach is dependent on having an accurate depth of the target (e.g., a fetus) within the body input in order to accurately calculate the blood oxygen saturation. This may pose a challenge in a clinical situation because measuring (via, for example, an ultrasound or Doppler device) depth of a target (e.g., a fetus, fetal head, or fetal back) within a body is open to clinical interpretation and may not always be reliable, especially when differences of as little as 5 mm can impact the accuracy of calculations. Furthermore, a target's depth that is measured by an ultrasound or Doppler device may not accurately reflect that path of the photons because, for example, the photons are a different signal type (i.e., optical) than the sound waves used by the ultrasound/Doppler device and/or the ultrasound/Doppler device is not positioned where the optical probe is positioned so the ultrasound/Doppler device may be imaging a portion of the surface tissue that is not coincident with the placement of the optical probe. In order to overcome the target depth requirement, machine learning may be deployed as a methodology to develop a model that augments the 2-layered description of the modified Beer Lambert Law to arrive at target SpO2 values without requiring target depth as an input. This model would then be able to accurately determine fetal oximetry values without requiring fetal depth.

Transdermal in vivo measurements of target (e.g., fetal) SpO2 levels may involve placing an optical probe (e.g., one or more light source(s) and photodetector(s)) on the skin of a patient (e.g., a pregnant woman), transmitting an optical signal into the skin of the patient, and collecting resulting optical signals emitted from the skin of the patient via, for example, backscattering and/or transmission through the patient's non-target tissue and target's tissue. In many cases, determining SpO2 involves calculating the amplitudes of the AC signals, normalizing them using (dividing by) the amplitude of the DC signals, and multiplying the normalized AC signals by a calibration factor that considers, for example, abdominal and/or fetal tissue scattering properties and/or fetal depth as a function of wavelength of the incident optical signal/light. However, this normalization methodology is, in many cases, too generic of an approach because, for example, the impact of the maternal/fetal tissue on the behavior of the incident light is uniform along the pathlength of the optical signal. However, in many cases, this is inaccurate due to one or more confounding influences of, for example, maternal tissue, maternal physiology, maternal movement, and/or fetal tissue/physiology/movement when the optical probe is being used and/or during measurement of fetal SpO2. Hence, a different normalization methodology for the AC signals may assist with more accurately calculating fetal SpO2.

In some embodiments, using one or more optical probe(s) that include multiple light sources (also called “sources” herein) and/or multiple photodetectors (also called “detectors” herein) that facilitate multiple sets of sources and detectors that have different source-detector distances (i.e., different distances between the source and detector) may provide inputs that can be used to compensate for confounding influences in some situations because, for example, a mean depth of penetration for the light into the patient's tissue (e.g., pregnant mammal's abdomen) gets larger as the source-detector separation increases. Shorter separations between the source and detector result in light that penetrates the tissue less deeply than light from larger separations between the source and detector. Use of signals detected when the source/detector distance is relatively short biases these measurement towards measuring only the patient's non-target (e.g., maternal, or abdominal) tissues (which are shallower than the target tissue), because the light detected by relatively close detectors only penetrates the patient's non-target tissue. In some cases, these detected signals may be called short separation signals.

Additionally, or alternatively, by using one or more probes with various source/detector distances both the patient's non-target-only signals (i.e., short separation signals) and a composite signal that includes light incident on both the patient's non-target and target tissue (sometimes referred to herein as a “composite signal”) may enable measurement and/or comparisons of variability of the patient's non-target and/or target tissue. In some instances, comparing detected signals from detectors with a short source/detector distance with detected signals from detectors with a longer source/detector distance may facilitate understanding of how the patient's non-target and/or target tissue my impact the behavior of light incident thereon. This information may be used, for example, to normalize AC signals, develop or adjust a calibration formula used to determine target SpO2, and/or develop or adjust a calibration factor used to determine target SpO2, which may make calculation of target SpO2 more accurate than previously used techniques.

In, for example, a transabdominal fetal oximetry context, using machine learning as a methodology to develop a model that augments the 2-layered (one maternal and one fetal) description of the modified Beer Lambert Law to arrive at fetal hemoglobin oxygen saturation values without requiring fetal depth as an input and/or incorporates confounding influences of maternal and fetal tissue when determining fetal SpO2 requires a large data set of fetal SpO2 values so that many different scenarios with different fetal depths and/or confounding factors may be understood and factored into a determination of fetal SpO2 in a particular situation. A data set of fetal oximetry and/or SpO2 values calculated using a model, or mathematical simulation, of the fetal and/or maternal tissue, which is sometimes referred to herein as “calculated fetal oximetry values” or “calculated fetal Spo2 values,” may be used to train and/or test a processor to determine fetal SpO2 values. In some instances, the model may be a physiological model of the fetus and mother, which in some cases may include static and time variant tissue layer properties of the fetus and/or pregnant mammal that may calculate how light may behave when transmitted and/or detected with various source-separation distances and light wavelengths. These calculated SpO2 values may, in some cases, represent a simulated light transmission time series data set (“also referred to herein as a “simulated light transmission data set”) that models the optical signals that may be detected by a detector over time and may thereby be available for analysis, manipulation, and input into machine learning models in a manner similar to, for example, actual light transmission data sets collected from a detector and/or actual fetal SpO2 values. In some embodiments, simulated light transmission data used to calculate fetal oximetry and/or SpO2 values may be determined and/or generated using machine learning equipment and/or techniques.

Additionally, or alternatively, the simulated light transmission data may be generated by software designed to build models and/or generate simulated data for light traveling through tissue and/or tissue model(s). Examples of this software are Monte Carlo simulations and Near Infrared Fluorescence and Spectral Tomography (NIRFAST, Dartmouth College, NH) software. Use of modeling software allows for models to be built that incorporate a variety of parameters such as wavelength of light used, DFP, source/detector distance, and/or maternal and/or fetal morphological, geometric and/or physiological parameters such as abdominal wall thickness and/or composition, tissue composition, tissue type, muscular state of the maternal uterus, maternal skin color, fetal skin color, and/or position on the fetus on which the light was incident. At times, the parameters of the data sets and/or inputs used to generate the models may be changed discretely, randomly, pseudo-randomly, and/or selected within a range and/or distribution of values. Additionally, or alternatively, combinations of input parameters may be used to generate the simulated signals. This approach and/or a combination of approaches may provide a random covering of the possible simulated light transmission data sets/time series and/or calculated fetal SpO2 values that may be used for training and testing the machine learning model. Additionally, or alternatively, features may be extracted from simulated light transmission data sets to be used as inputs to the machine learning architecture or models. Examples of potential features that may be extracted from simulated light transmission data sets are correlation amplitudes, FFTs, time of flight for photons exiting the maternal abdomen, DC levels, AC levels or other post-processed signal descriptors.

Possible uses and/or advantages of the present invention include, but are not limited to, facilitation of perturbation analysis of the data sets whereby one variable (e.g., maternal heart rate, fetal heart rate, fetal distance, source/detector distance) is changed at a time to determine an impact (if at all) on the calculated fetal SpO2 values. This is a substantial advantage over experimentally determined data sets, or calculated fetal SpO2 values, because it is difficult, in real life, to control only one factor at a time because, often times, multiple factors change at unpredictable rates/times with in vivo situations.

Additionally, or alternatively, the present invention may be used to perform sensitivity analysis, which may allow for changing multiple variables/parameters used to generate the models and/or simulated light transmission data sets so that, for example, the results (e.g., calculated fetal SpO2 values) may be evaluated for accuracy and/or to determine how multiple variables may interact with one another to vary calculated fetal SpO2 values. Model variables that may be modified to perform sensitivity analysis include, but are not limited to, noise, wavelength of light used, DFP, source/detector distance, and/or maternal and/or fetal morphological, geometric and/or physiological parameters such as abdominal wall thickness and/or composition, tissue composition, tissue type, muscular state of the maternal uterus, maternal skin color, fetal skin color, and/or position on the fetus on which the light was incident.

Additionally, or alternatively, an advantage of the present invention is that use of simulated light transmission data sets and/or fetal SpO2 values calculated using the simulated light transmission data sets to train a simulate fetal oximetry model, or teach the machine, reduces the number of experimentally, or measured, in vivo light transmission data sets and/or fetal SpO2 values that are necessary to arrive at an accurately trained model. This, in turn, reduces the need for a very large and difficult to obtain data set of actual fetal SpO2 values determined using measured/in vivo data (e.g., a blood gas analysis).

In some embodiments, the simulated and/or in vivo fetal oximetry models disclosed herein may be augmented with additional data and/or correlations, thereby generating augmented simulated fetal oximetry models and/or augmented in vivo fetal oximetry models. The augmented simulated fetal oximetry models and/or augmented in vivo fetal oximetry models (also referred to herein as a “augmented fetal oximetry model” and/or “personalized in vivo fetal oximetry model”) may incorporate, for example, simulated and/or measured physiological indicators, measurements, values, and/or parameters for the pregnant mammal and/or fetus and/or correlations between the simulated and/or measured physiological indicators and/or parameters and a medical condition and/or diagnosis. These correlations may be based on a single value for one or more physiological indicator(s) and/or a set of values for the one or more physiological indicator(s) taken over time (e.g., 5, 10, 20, or 30 minutes). For example, values for the physiological indicator of fetal heart rate for a single time period (e.g., 15, 30, or 60 seconds) may be correlated with one or more evaluations or diagnosis such as “within normal range,” “abnormally low,” and “abnormally high” and values for a plurality of physiological indicators measured over time (e.g., 10, 20, or 30 minutes) may be correlated with the “within normal range,” “abnormally low,” and “abnormally high” fetal heart rate evaluations as well as indications that are time-dependent such as trends (e.g., decreasing fetal heart rate over time), dramatic changes in fetal heart rate values over time, or a large phase difference, which may be indicators of fetal distress. Exemplary simulated and/or measured physiological indicators, measurements, values, and/or parameters for the pregnant mammal and/or fetus include, but are not limited to, heart rate, pulse, ECG information, uterine tone, uterine contraction timing and/or duration, temperature, oximetry values, pulse oximetry values, and tissue oximetry values.

The in vivo fetal oximetry models and/or augmented in vivo fetal oximetry models disclosed herein may be used to evaluate and/or predict a condition (e.g., distress or a lack of distress) of the pregnant mammal and/or fetus in leu of and/or in addition to determining a fetal oxygenation level. These determinations may be provided to a user in a variety of fashions that include, but are not limited to, a score and/or alarm condition that factors in, for example, fetal oxygenation values and/or levels, a duration of time a fetus has a determined oxygenation level, fetal heart rate, fetal ECG values/measurements, and/or a correspondence between fetal heart rate and maternal contractions. These determinations may be used by a clinician to direct care (e.g., perform a Cesarian section, administer medication, or continue to watch and monitor fetal development and/or a labor and delivery process) for the pregnant mammal and/or fetus.

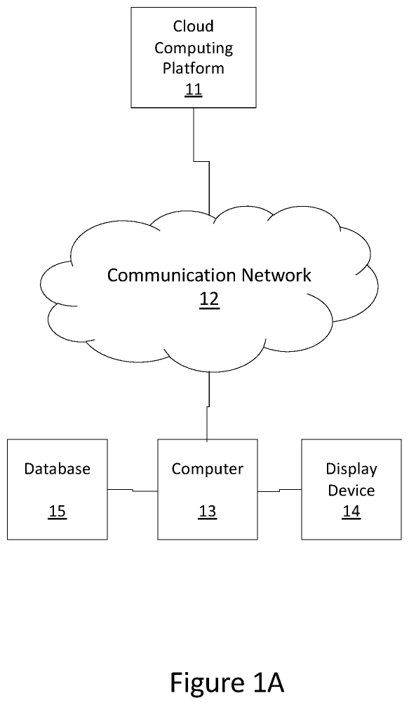

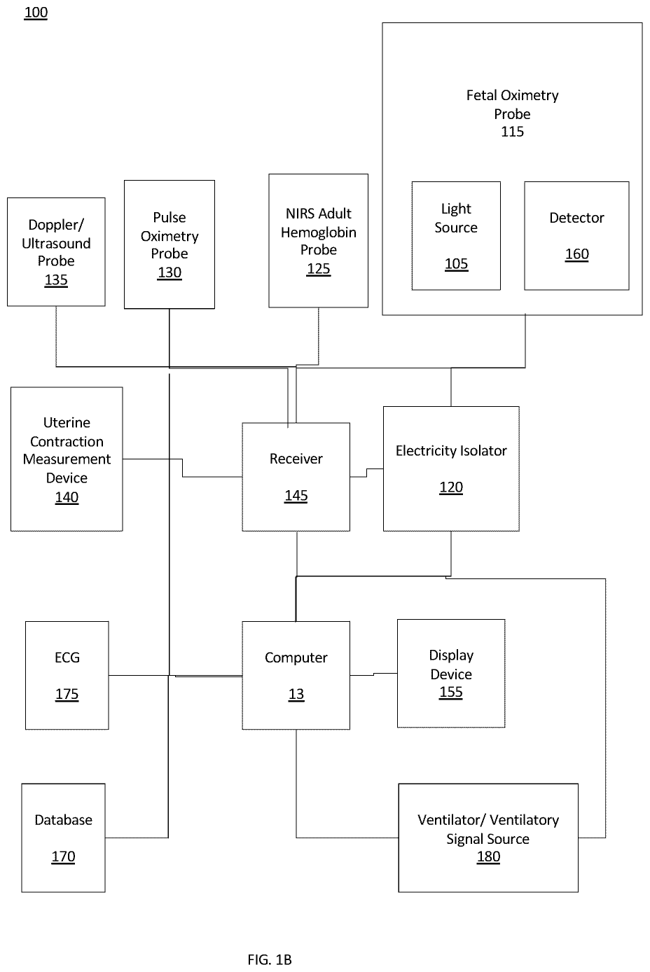

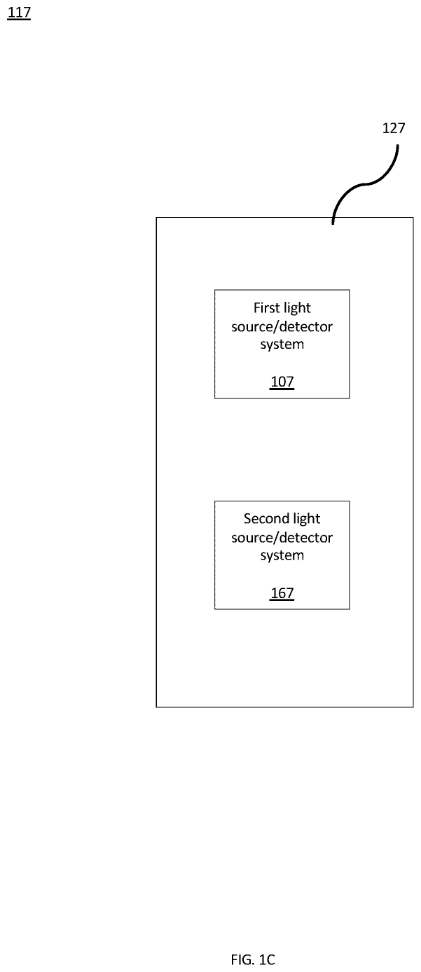

FIG. 1A provides an exemplary system 10 for using machine learning to develop a simulated fetal oximetry model and/or simulated augmented fetal oximetry model and/or an in vivo fetal oximetry model and/or augmented in vivo fetal oximetry model as disclosed herein. In some cases, the developed simulated fetal oximetry model and/or simulated augmented fetal oximetry model and/or an in vivo fetal oximetry model and/or augmented in vivo fetal oximetry model may compensate for one or more physio-optical influences that occur when performing transabdominal fetal oximetry. System 10 includes a cloud computing platform 11, a communication network 12, a computer 13, a display device 14, and a database 15. In many instances, communication network 12 is the Internet. The components of system 10 may be coupled together via wired and/or wireless communication links. In some instances, wireless communication of one or more components of system 10 may be enabled using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below.

Cloud computing platform 11 may be any cloud computing platform 11 configured to run a machine learning program and/or support a machine learning architecture such as TensorFlow. Exemplary cloud computing platforms include, but are not limited to, Amazon Web Service (AWS), Rackspace, and Microsoft Azure. Exemplary machine learning architectures include neural networks, artificial neural networks, Bayesian networks, and/or software or hardware that utilizes artificial intelligence.

Computer 13 may be configured to act as a communication terminal to cloud computing platform 11 via, for example, communication network 12 and may facilitate provision of the results machine learning calculations (e.g., training and/or testing of a simulated fetal oximetry model and/or simulated augmented fetal oximetry model, tuning of a simulated fetal oximetry model and/or simulated augmented fetal oximetry model, training and/or testing of a in vivo fetal oximetry model and/or augmented in vivo fetal oximetry model, and/or tuning of the in vivo fetal oximetry model and/or augmented in vivo fetal oximetry model) performed on cloud computing platform 11 to display device 155. Exemplary computers 13 include desktop and laptop computers, servers, tablet computers, personal electronic devices, mobile devices (e.g., smart phones), and the like. Exemplary display devices 155 are computer monitors, tablet computer devices, and displays provided by one or more of the components of system 10. In some instances, display device 155 may be resident in computer 13. Computer 13 may be communicatively coupled to database 15, which may be configured to store information (e.g., simulated optical inputs, simulated light transmission data sets, levels of a simulated fetal oximetry model and/or simulated augmented fetal oximetry model, simulated and/or calculated fetal oximetry values, in vivo light transmission data sets, levels of an in vivo fetal oximetry model and/or augmented in vivo fetal oximetry model, model testing results, etc.), or inputs, used for machine learning and/or sets of instructions for computer 13 and/or cloud computing platform 11.

FIG. 1B is a block diagram of an exemplary system 100 for measuring in vivo light transmission data, measuring in vivo fetal oximetry values, and/or determining in vivo fetal oximetry values. In some embodiments, system 100 and/or a component thereof, such as computer 13, may be communicatively coupled to system 10, or a component thereof such as communication network 12 and/or cloud computing platform 11. The components of system 100 may be coupled together via wired and/or wireless communication links. In some instances, wireless communication of one or more components of system 100 may be enabled by using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below.

System 100 includes a light source 105 and a detector 160 that, at times, may be housed in a single housing, which may be referred to as a fetal oximetry probe 115. Fetal oximetry probe 115 may be internally (e.g., transcervical, transvaginal, transurethral, and/or transuteral) placed and/or be non-invasively placed on the abdomen of the pregnant mammal. Light source 105 may include a single, or multiple light sources and detector 160 may include a single, or multiple detectors.

Light sources 105 may transmit light at light of one or more wavelengths, including NIR, into the pregnant mammal's abdomen. Typically, the light emitted by light sources 105 will be focused or emitted as a narrow beam to reduce spreading of the light upon entry into the pregnant mammal's abdomen. Light sources 105 may be, for example, a LED, and/or a LASER, a tunable light bulb and/or a tunable LED that may be coupled to a fiber optic cable. On some occasions, the light sources may be one or more fiber optic cables optically coupled to a laser and arranged in an array. In some instances, the light sources 105 may be tunable or otherwise user configurable while, in other instances, one or more of the light sources may be configured to emit light within a pre-defined range of wavelengths. Additionally, or alternatively, one or more filters (not shown) and/or polarizers may filter/polarize the light emitted by light sources 105 to be of one or more preferred wavelengths. These filters/polarizers may also be tunable or user configurable.

An exemplary light source 105 may have a relatively small form factor and may operate with high efficiency, which may serve to, for example, conserve space and/or limit heat emitted by the light source 105. In one embodiment, light source 105 is configured to emit light in the range of 770-850 nm. Exemplary flux ratios for light sources include but are not limited to a luminous flux/radiant flux of 50-260 mW, a total radiant flux of 50-550 mW and a power rating of 0.3 W-3.5 W.

Detector 160 may be configured to detect a light signal emitted from the pregnant mammal and/or the fetus via, for example, transmission and/or back scattering. Detector 160 may convert this light signal into an electronic signal, which may be communicated to a computer or processor and/or an on-board transceiver that may be capable of communicating the signal to the computer/processor. This emitted light might then be processed in order to determine how much light, at various wavelengths, passes through the fetus and/or is reflected and/or absorbed by the fetal oxyhemoglobin and/or de-oxyhemoglobin so that a fetal hemoglobin oxygen saturation level may be determined. This processing will be discussed in greater detail below. In some embodiments, detector 160 may be configured to detect/count single photons. At times, the optical signals detected by detector 160 and converted into an electronic signal corresponding to the detected optical signal may be referred to herein as measured, or in vivo, light transmission data and/or a detected electronic signal.

Exemplary detectors include, but are not limited to, cameras, traditional photomultiplier tubes (PMTs), silicon PMTs, avalanche photodiodes, and silicon photodiodes. In some embodiments, the detectors will have a relatively low cost (e.g., $50 or below), a low voltage requirement (e.g., less than 100 volts), and non-glass (e.g., plastic) form factor. In other embodiments, (e.g., contactless pulse oximetry) a sensitive camera may be deployed to receive light emitted by the pregnant mammal's abdomen. For example, detector 160 may be a sensitive camera adapted to capture small changes in fetal skin tone caused by changes in cardiovascular pressure associated with fetal myocardial contractions. In these embodiments, detector 160 and/or fetal oximetry probe 115 may be in contact with the pregnant mammal's abdomen, or not, as this embodiment may be used to perform so-called contactless pulse oximetry. In these embodiments, light sources 105 may be adapted to provide light (e.g., in the visible spectrum, near-infrared, etc.) directed toward the pregnant mammal's abdomen so that the detector 160 is able to receive/detect light emitted by the pregnant mammal's abdomen and fetus. The emitted light captured by detector 160 may be communicated to computer 13 for processing to convert the images to a measurement of fetal hemoglobin oxygen saturation according to, for example, one or more of the processes described herein.

A fetal oximetry probe 115, light source 105, and/or detector 160 may be of any appropriate size and, in some circumstances, may be sized so as to accommodate the size of the pregnant mammal using any appropriate sizing system (e.g., waist size and/or small, medium, large, etc.). Exemplary lengths for a fetal oximetry probe 115 include a length of 4 cm-40 cm and a width of 2 cm-10 cm. In some circumstances, the size and/or configuration of a fetal oximetry probe 115, or components thereof, may be responsive to skin pigmentation of the pregnant mammal and/or fetus. In some instances, the fetal oximetry probe 115 may be applied to the pregnant mammal's skin via tape or a strap that cooperates with a mechanism (e.g., snap, loop, etc.) (not shown). In some instances, fetal oximetry probe 115 may act to pre-process or filter detected signals.

System 100 includes a number of optional independent sensors/probes designed to monitor various aspects of maternal and/or fetal health and may be in contact with a pregnant mammal. These probes/sensors are a NIRS adult hemoglobin probe 125, a pulse oximetry probe 130, a Doppler and/or ultrasound probe 135, and a uterine contraction measurement device 140. Not all embodiments of system 100 will include all of these components. In some embodiments, system 100 may also include an electrocardiogramachine (not shown) that may be used to determine the pregnant mammal's and/or fetus's heart rate and/or an intrauterine pulse oximetry probe (not shown) that may be used to determine the fetus's heart rate. The Doppler and/or ultrasound probe 135 may be configured to be placed on the abdomen of the pregnant mammal and may be of a size and shape that approximates a silver U.S. dollar coin and may provide information regarding fetal position, orientation, and/or heart rate. Pulse oximetry probe 130 may be a conventional pulse oximetry probe placed on pregnant mammal's hand and/or finger to measure the pregnant mammal's hemoglobin oxygen saturation. NIRS adult hemoglobin probe 125 may be placed on, for example, the pregnant mammal's 2nd finger or earlobe and may be configured to, for example, use near infrared spectroscopy to calculate the ratio of adult oxyhemoglobin to adult de-oxyhemoglobin. NIRS adult hemoglobin probe 125 may also be used to determine the pregnant mammal's heart rate.

Optionally, system 100 may include a uterine contraction measurement device 140 configured to measure the strength and/or timing of the pregnant mammal's uterine contractions. In some embodiments, uterine contractions will be measured by uterine contraction measurement device 140 as a function of pressure (e.g., measured in e.g., mmHg) over time. In some instances, the uterine contraction measurement device 140 is and/or includes a tocotransducer, which is an instrument that includes a pressure-sensing area that detects changes in the abdominal contour to measure uterine activity and, in this way, monitors frequency and duration of contractions. Additionally, or alternatively, uterine contraction measurement device 140 may employ electromyography EMG) to measure electrical activity from the uterine muscle to detect one or more characteristics of uterine contractions during a labor and delivery process.

In another embodiment, uterine contraction measurement device 140 may be configured to pass an electrical current through the pregnant mammal and measure changes in the electrical current as the uterus contracts. Additionally, or alternatively, uterine contractions may also be measured via near infrared spectroscopy using, for example, light received/detected by detector 160 because uterine contractions, which are muscle contractions, are oscillations of the uterine muscle between a contracted state and a relaxed state. Oxygen consumption of the uterine muscle during both of these stages is different and these differences may be detectable using NIRS.

Measurements and/or signals from NIRS adult hemoglobin probe 125, pulse oximetry probe 130, Doppler and/or ultrasound probe 135, and/or uterine contraction measurement device 140 may be communicated to receiver 145 for communication to computer 13 and display on display device 155 and, in some instances, may be considered secondary signals. As will be discussed below, measurements provided by NIRS adult hemoglobin probe 125, pulse oximetry probe 130, a Doppler and/or ultrasound probe 135, uterine contraction measurement device 140 may be used in conjunction with fetal oximetry probe 115 to isolate a fetal pulse signal and/or fetal heart rate from a maternal pulse signal and/or maternal heart rate. Receiver 145 may be configured to receive signals and/or data from one or more components of system 100 including, but not limited to, fetal oximetry probe 115, NIRS adult hemoglobin probe 125, pulse oximetry probe 130, Doppler and/or ultrasound probe 135, and/or uterine contraction measurement device 140. Communication of receiver 145 with other components of system may be made using wired or wireless communication.

In some instances, one or more of NIRS adult hemoglobin probe 125, pulse oximetry probe 130, a Doppler and/or ultrasound probe 135, uterine contraction measurement device 140 may include a dedicated display that provides the measurements to, for example, a user or medical treatment provider. It is important to note that not all of these probes may be used in every instance. For example, when the pregnant mammal is using fetal oximetry probe 115 in a setting outside of a hospital or treatment facility (e.g., at home or work) then, some of the probes (e.g., NIRS adult hemoglobin probe 125, pulse oximetry probe 130, a Doppler and/or ultrasound probe 135, uterine contraction measurement device 140) of system 100 may not be used.

In some instances, receiver 145 may be configured to process or pre-process received signals so as to, for example, make the signals compatible with computer 13 (e.g., convert an optical signal to an electrical signal), improve signal to noise ratio (SNR), amplify a received signal, etc. In some instances, receiver 145 may be resident within and/or a component of computer 13. In some embodiments, computer 13 may amplify or otherwise condition the received detected signal so as to, for example, improve the signal-to-noise ratio.

Receiver 145 may communicate received, pre-processed, and/or processed signals to computer 13. Computer 13 may act to process the received signals, as discussed in greater detail below, and facilitate provision of the results to a display device 155. Exemplary computers 13 include desktop and laptop computers, servers, tablet computers, personal electronic devices, mobile devices (e.g., smart phones), and the like. Exemplary display devices 155 are computer monitors, tablet computer devices, and displays provided by one or more of the components of system 100. In some instances, display device 155 may be resident in receiver 145 and/or computer 13. Computer 13 may be communicatively coupled to database 170, which may be configured to store information regarding physiological characteristic and/or combinations of physiological characteristic of pregnant mammals and/or their fetuses, impacts of physiological characteristic on light behavior, information regarding the calculation of hemoglobin oxygen saturation levels, calibration factors, calibration formulas, calibration curves, and so on.

In some embodiments, a pregnant mammal may be electrically insulated from one or more components of system 100 by, for example, an electricity isolator 120. Exemplary electricity insulators 120 include devices configured to ensure leakage currents remain at a safe level and may deploy magnetic and/or optical devices and/or techniques to insulate against unsafe leakage of electrical currents. System 100 may include a plurality of electricity insulators 120 that may be positioned in a plurality of locations.

In some embodiments, system 100 may include an electro-cardiogram (ECG) machine 175 configured to ascertain characteristics of the pregnant mammal's heart rate and/or pulse and/or measure same. These characteristics may be used as, for example, a secondary signal and/or maternal heart rate signal as disclosed herein.

In some embodiments, system 100 may include a ventilatory/respiratory signal source 180 that may be configured to monitor the pregnant mammal's respiratory rate and provide a respiratory signal indicating the pregnant mammal's respiratory rate to, for example, computer 13. Additionally, or alternatively, ventilatory/respiratory signal source 180 may be a source of a ventilatory signal obtained via, for example, cooperation with a ventilation machine. Exemplary ventilatory/respiratory signal sources 180 include, but are not limited to, a carbon dioxide measurement device, a stethoscope and/or electronic acoustic stethoscope, a device that measures chest excursion for the pregnant mammal, and a pulse oximeter. A signal from a pulse oximeter may be analyzed to determine variations in the PPG signal that may correspond to respiration for the pregnant mammal. Additionally, or alternatively, ventilatory/respiratory signal source 180 may provide a respiratory signal that corresponds to a frequency with which gas (e.g., air, anesthetic, etc.) is provided to the pregnant mammal during, for example, a surgical procedure. This respiratory signal may be used to, for example, determine a frequency of respiration for the pregnant mammal.

In some embodiments, system 100 may include a timestamping device 185. Timestamping device 185 may be configured to timestamp a signal provided by, for example, fetal oximetry probe 115, Doppler/ultrasound probe 135, pulse oximetry probe 130, NIRS adult hemoglobin probe, uterine contraction measurement device 140, ECG 175, and/or ventilatory/respiratory signal source 180 with a timestamp that represents, for example, an event (e.g., time, or t, =0, 10, 20, etc.) and/or chronological time (e.g., date and time). Timestamping device 185 may time stamp a signal via, for example, introducing a ground signal into system 100 that may simultaneously, or nearly simultaneously, interrupt or otherwise introduce a stamp or other indicator into a signal generated by one or more of, for example, fetal oximetry probe 115, Doppler/ultrasound probe 135, pulse oximetry probe 130, NIRS adult hemoglobin probe, uterine contraction measurement device 140, ECG 175, and/or ventilatory/respiratory signal source 180. Additionally, or alternatively, timestamping device 185 may time stamp a signal via, for example, introducing an optical signal into system 100 that may simultaneously, or nearly simultaneously, interrupt or otherwise introduce a stamp or other indicator into a signal generated by one or more of, for example, fetal oximetry probe 115, pulse oximetry probe 130, NIRS adult hemoglobin probe, uterine contraction measurement device 140. Additionally, or alternatively, timestamping device 185 may time stamp a signal via, for example, introducing an acoustic signal into system 100 that may simultaneously, or nearly simultaneously, interrupt or otherwise introduce a stamp or other indicator into a signal generated by one or more of, for example, fetal oximetry probe 115, Doppler/ultrasound probe 135, and/or ventilatory/respiratory signal source 180.

A timestamp generated by timestamping device 185 may serve as a simultaneous, or nearly simultaneous starting point, or benchmark, for the processing, measuring, synchronizing, correlating, and/or analyzing of a signal from, for example, fetal oximetry probe 115, Doppler/ultrasound probe 135, pulse oximetry probe 130, NIRS adult hemoglobin probe, uterine contraction measurement device 140, ECG 175, and/or ventilatory/respiratory signal source 180. In some instances, a time stamp may be used to relate and/or synchronize two or more signals generated by, for example, fetal oximetry probe 115, Doppler/ultrasound probe 135, pulse oximetry probe 130, NIRS adult hemoglobin probe, uterine contraction measurement device 140, ECG 175, and/or ventilatory/respiratory signal source 180 so that, for example, they align in the time domain.

FIG. 1C is a block diagram of an exemplary fetal oximetry probe 117 that may be used with system 100 in addition to, or instead of, fetal oximetry probe 115. Fetal oximetry probe 117 includes a first light source/detector system 107 and a second light source/detector system 167 housed within a housing 127 that may be configured to enable use of the fetal oximetry probe 117. First light source/detector system 107 and second light source/detector system 167 may be configured in a manner that is similar to, or different from, one another. For example, first light source/detector system 107 may be configured as a frequency-domain measurement system and second light source/detector system 167 may be configured as a near infrared spectroscopy system. Additionally, or alternatively, first light source/detector system 107 may be configured as a system that measures a time of flight of photons projected into the pregnant mammal's abdomen and returning to one or more detectors like detectors 160. On some occasions, the frequency-domain and/or time of flight measurements may be used to, for example, determine optical properties (e.g., scattering and/or absorption coefficients) of maternal and/or fetal tissue. Relative positions of the first light source/detector system 107 and second light source/detector system 167 may be known so that, for example, data received via by first light source/detector system 107 may be used to validate and/or further analyze data received via second light source/detector system 167.

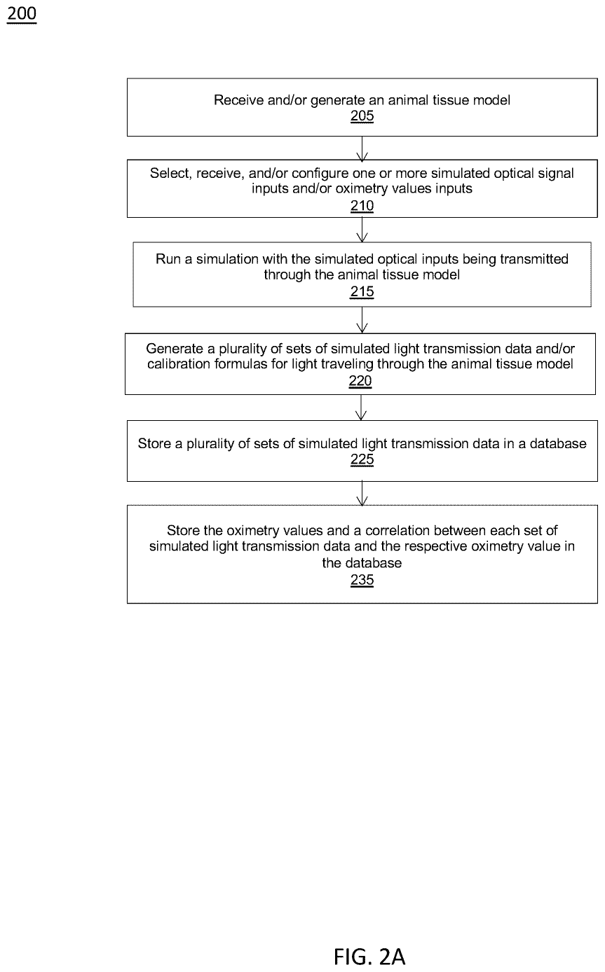

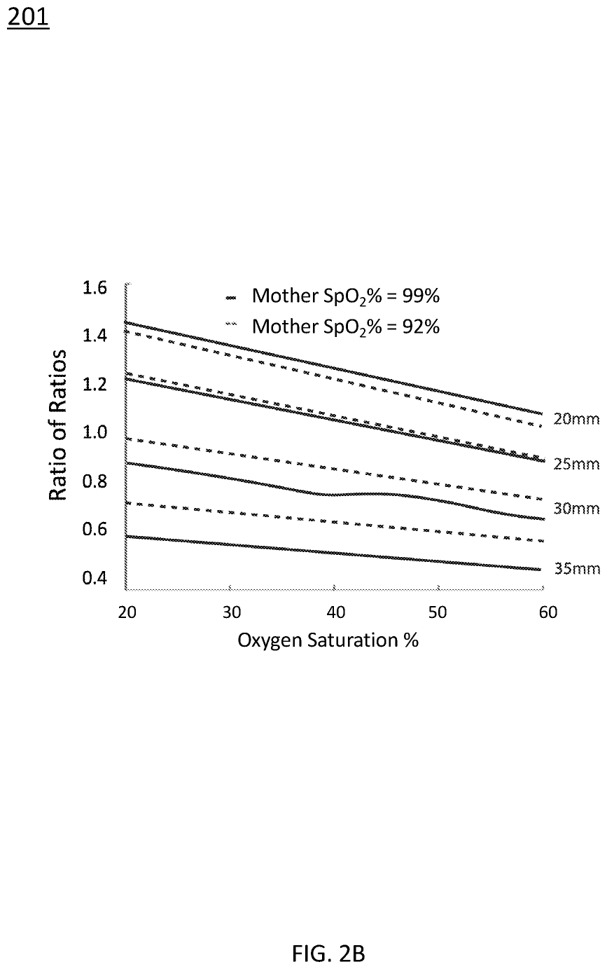

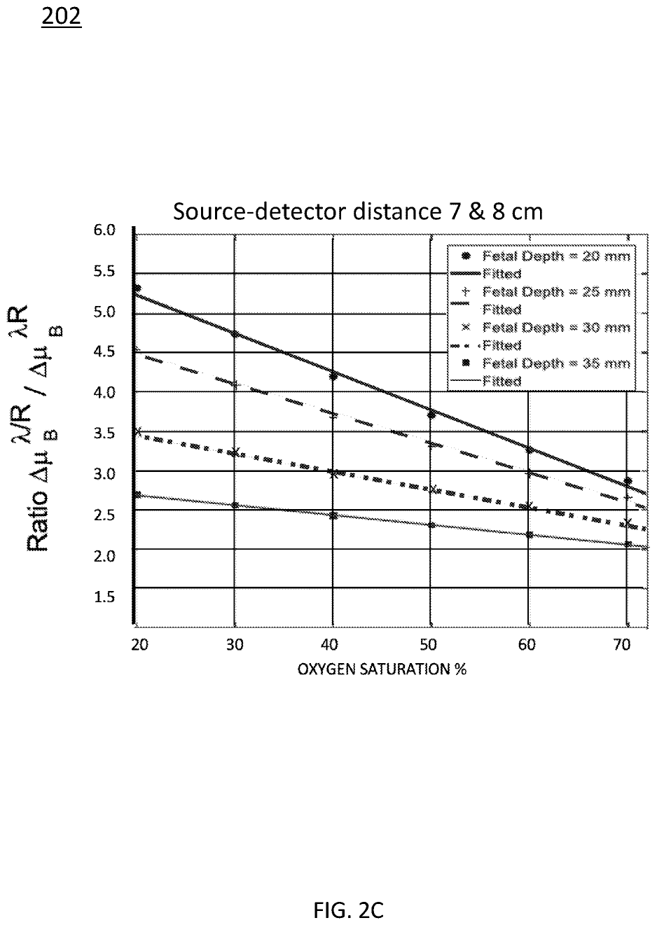

FIG. 2A is a flowchart showing an exemplary process 200 for generating a plurality of sets of simulated light transmission data and corresponding oximetry values using a computer-generated, or simulated, model of animal tissue. Process 200 may be executed by, for example, system 100, 10, and/or components thereof.

Initially, in step 205, a two and/or three-dimensional model of a portion of animal tissue may be generated and/or received. The layers of the model may each have different optical properties such as absorption and/or reflection characteristics, blood saturation characteristics, in some cases, these optical properties may be dictated by properties of the tissue such as lipid content, water content, density, and/or tissue type. When process 200 is executed multiple times, one or more aspects of the model received and/or generated in step 205 may change.

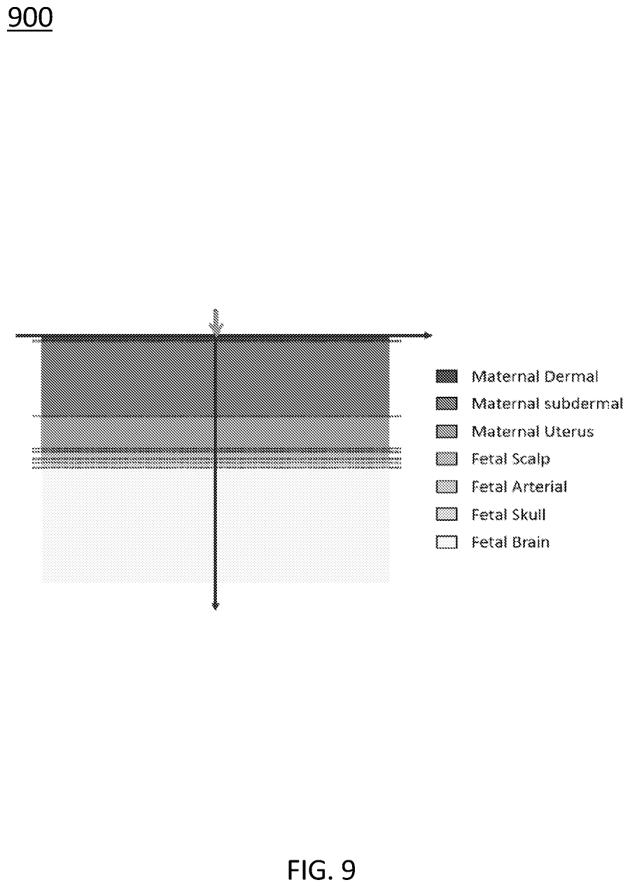

The model(s) of step 205 may include, for example, 1-8 layers of tissue; some of which may be fetal tissue. In many embodiments, the modeled tissue may include at least two layers one of which corresponds to maternal tissue and the other layer corresponds to fetal tissue. FIG. 9 provides an image of an exemplary seven-layer two-dimensional model 900 of a pregnant mammal's abdomen and her fetus that may be received and/or generated at step 205. The seven layers of two-dimensional model 900 are 1) maternal dermal, 2) maternal subdermal, 3) maternal uterus, 4) fetal scalp, 5) fetal arterial, 6) fetal skull, and 7) fetal brain. Each of these layers may have different optical properties based on, for example, light wavelength, light intensity, tissue layer composition, tissue layer thickness, and/or tissue layer geometry.

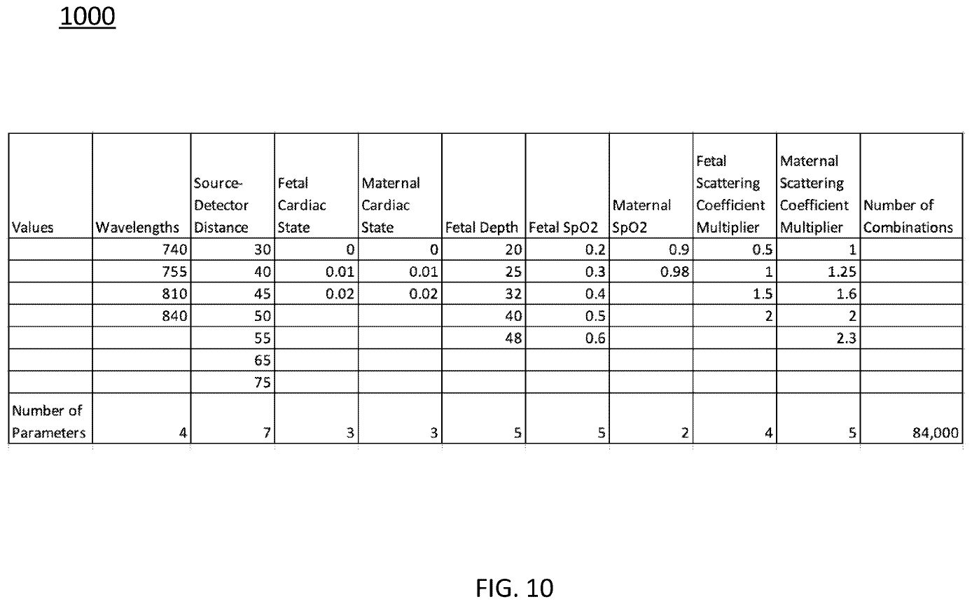

In some cases, execution of step 205 may also include receipt and/or selection of parameters or rules for the model, some of which may be machine learning inputs and/or optical properties of one or more layers of the model. FIG. 10 provides a table of exemplary parameters 1000 that may be used to generate a model in step 205 and/or may govern one or more aspects and/or functions of a model that is received. When a generated model is received in step 205 (as opposed to generated), a table like the table of FIG. 10 may also be received so that one or more parameters of the model may be understood. In some embodiments, one or more parameters of a model may dictate behavior of one or more simulated light transmission data sets.

The matrix of properties of table 1000 may be used to generate a two- and/or three-dimensional model, like exemplary model 900, of the pregnant mammal and/or fetus, which may then be used to generate a plurality of simulated light transmission data sets. More specifically, table 1000 provides exemplary values for wavelength of light emitted by the source, a distance between the source and a detector, fetal cardiac state, maternal cardiac state, fetal depth, fetal SpO2, maternal SpO2, fetal scattering coefficient multiplier, and maternal scattering coefficient multiplier. Additionally, or alternatively, other parameters may be used to generate the models such as tissue composition (e.g., lipid content, water content, muscle cell content, etc.), noise, ambient light, geometry of the portion of the human body being modeled, fetal and maternal cross correlation with heartbeats, DC level, normalization ratios, and/or fetal depth.

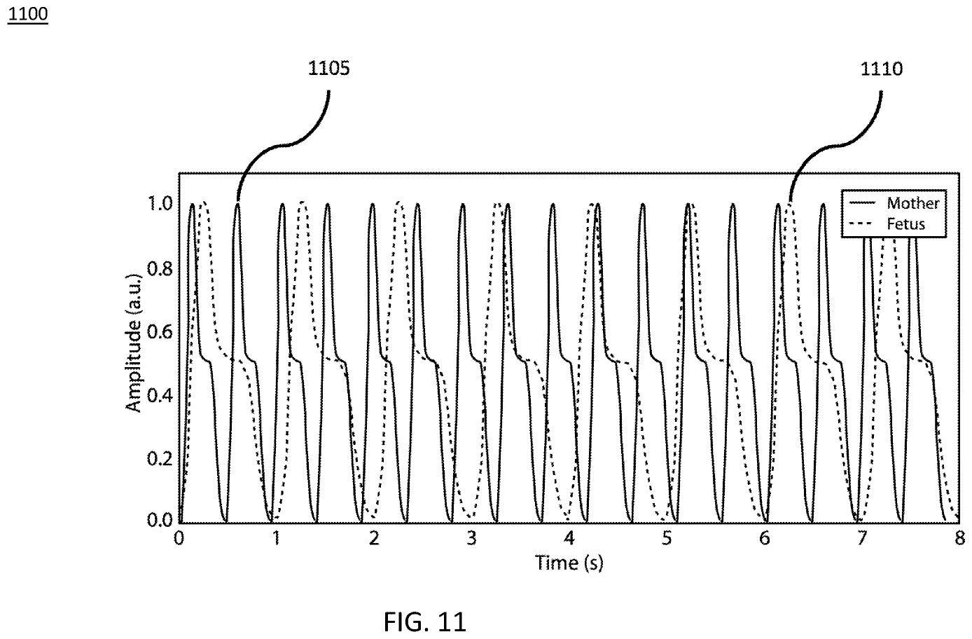

In some embodiments, a photoplethysmogram (PPG) modulated signal may be integrated into one or more of the models generated and/or received in step 205 to simulate cardiac cycles for the pregnant mammal and/or fetus. The PPG modulated signal may have, for example, a variable 1% to 2% change in systolic blood volume for the pregnant mammal and/or fetus. FIG. 11 provides a graph 1100 that plots a simulated fetal and maternal PPG signals over time in seconds, wherein a PPG signal for the mother/pregnant mammal 1105 is shown in black and a PPG signal for the fetus 1110 is shown in grey. In some embodiments, noise and/or a confounding factor may be added to the PPG signal for the fetus 1110 and/or pregnant mammal 1105 as part of, for example, perturbation analysis using the model(s).

In step 210, one or more simulated optical inputs for the generation of simulated light transmission data as simulated light corresponding to the simulated optical inputs travels through the model of step 205 may be selected, received, and/or configured. Exemplary simulated optical inputs include, but are not limited to, simulated light wavelength, intensity, modulation of the light (e.g., a duration of successive light pulses), scattering coefficients, absorption coefficients, fetal depth, maternal skin color, fetal skin color, maternal and/or fetal tissue layer composition (e.g., skin, adipose, muscle) and relative thicknesses of the tissue layers for the maternal and/or fetal tissue, and/or a range of wavelengths. In some cases, the simulated optical inputs will be for the generation of simulated infra-red and/or near infra-red light. Additionally, or alternatively, oximetry values inputs may also be received, selected, and/or configured in step 210. The oximetry value inputs may be, for example, a percent of hemoglobin saturated with oxygen (i.e., hemoglobin oxygen saturation percent), a relative oximetry value, and/or a ratio of oxygenated hemoglobin compared with deoxygenated hemoglobin.

Next, in step 215, a simulation using the model and simulated optical inputs of steps 205 and 210, respectively, may be run wherein simulated light is transmitted through the model and “detected” by a simulated photodetector. A result of execution of step 215 is the generation of a set of simulated light transmission data and/or calibration formulas for light traveling through the animal tissue model (step 220). In some cases, a set of simulated light transmission data may correspond to simulated light being transmitted through the model for a period of time (e.g., 15, 30, or 60 seconds; 1, 5, or 10 minutes). At times, the calibration formulas may relate, for example, a ratio of ratios (R) and/or an optical density of tissue with fetal oximetry values. On some occasions, a plurality (e.g., 100-100,000) of calibration formulas may be generated that incorporate various factors and/or inputs regarding light (e.g., wavelength and/or intensity) that may be simulated to travel through the animal model; geometrical properties (e.g., distance light travels (e.g., fetal depth or DFP), a shape of tissue within the animal tissue model and/or a thickness of tissue within the animal tissue model; optical properties of the animal model (e.g., scattering coefficient and absorption coefficient); and/or physiological properties of the modeled maternal and/or fetal tissue (e.g., maternal hemoglobin oxygen saturation levels and/or skin color). Steps 215 and 220 may be executed a plurality (e.g., 50,000; 100,000; 500,000; 1,000,000; 5,000,000) of times thereby generating a plurality of sets of simulated light transmission data and/or calibration formulas. The plurality of simulated light transmission data sets and/or calibration formulas may then be stored in a database (step 225) like database 15 and/or 170. In some embodiments, simulated light transmission data sets may include a simulation of an electronic signal that may be “detected” by a photodetector responsively to detecting light (in this case, the simulated optical input) as it travels through the animal model.

Following step 225, the oximetry values and/or correlations between each set of simulated light transmission data and/or calibration formula and it's respective oximetry value may be stored in a database (step 230) like database 15 and/or 170.

FIG. 2B is a graph 201 that plots exemplary relationships between a ratio of ratios (R) and a hemoglobin oxygen saturation percentage of a fetus for various fetal depths along with best fit curves that may be calculated/determined via process 200. More particularly, graph 201 plots how a ratio of ratios (R) and, in particular an R value for fetal a fetus, is related with a hemoglobin oxygen saturation percentage of a fetus for fetal depths of 20 mm, 25 mm, 30 mm, and 35 mm when the maternal SpO2% is 99% (solid line) and 92% (broken line) along with a corresponding best-fit curve for each fetal depth. A formula defining the best-fit line may, in some cases, be a calibration formula for use with, for example, one or more of the models disclosed herein. On some occasions, the best-fit line(s) of graph 201 may be calibration curve(s).

FIG. 2C is a graph 202 that plots exemplary relationships between a ratio of a change in an absorption coefficient for an infrared wavelength of light, or first wavelength, divided by a ratio of a change in an absorption coefficient for a red wavelength of light, or a second wavelength, is related to a hemoglobin oxygen saturation percentage of a fetus for fetal depths of 20 mm, 25 mm, 30 mm, and 35 mm along with a corresponding best-fit curve for each fetal depth that may be calculated/determined via process 200. A formula defining the best-fit line(s) of graph 202 may, in some cases, be a calibration formula for use with, for example, one or more of the models disclosed herein. On some occasions, the best-fit line(s) of graph 202 may be calibration curve(s).

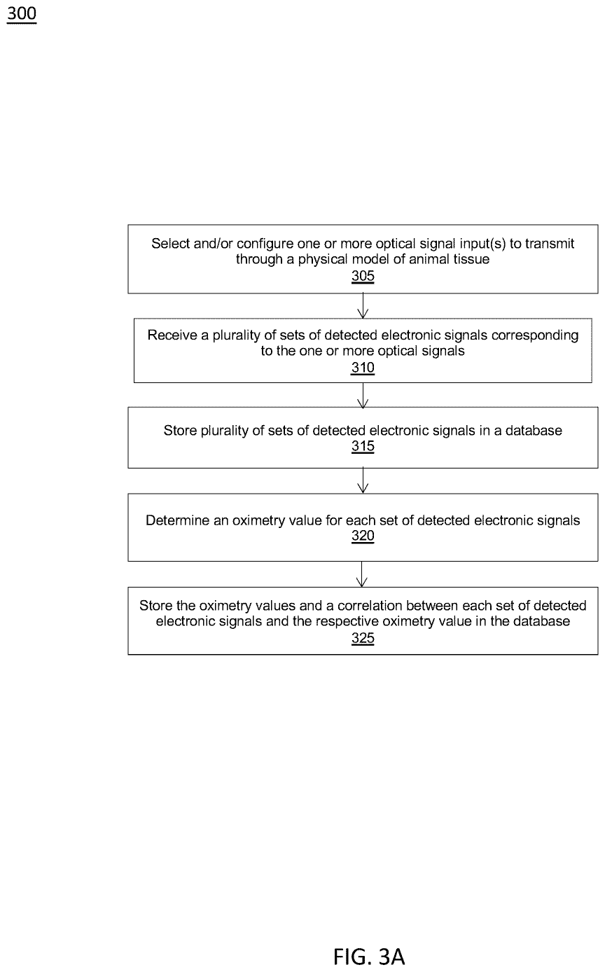

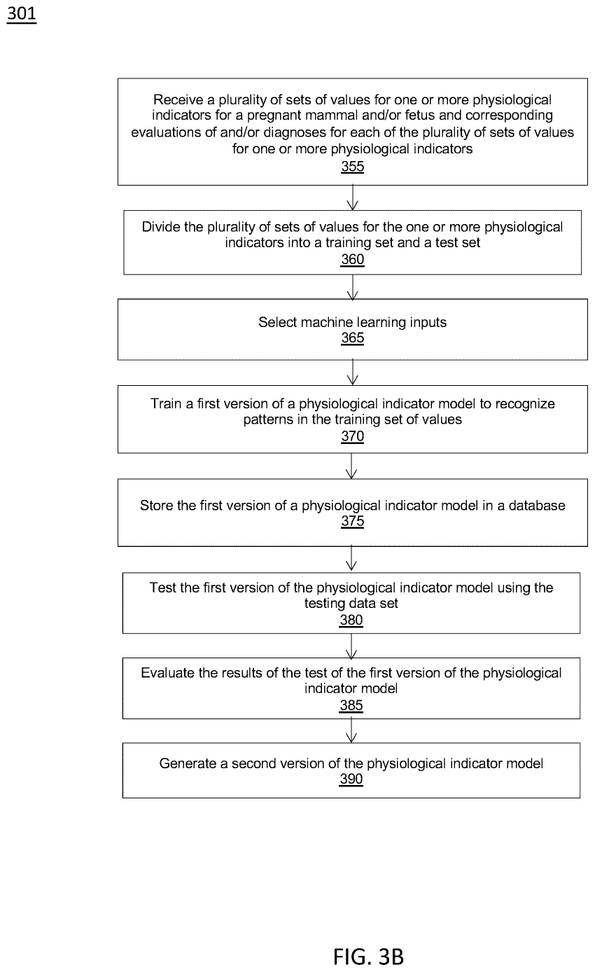

FIG. 3A is a flowchart showing an exemplary process 300 for generating a plurality of sets of simulated light transmission data and corresponding oximetry values using light transmitted through a physical model of animal tissue. Portions of process 300 may be executed by, for example, system 100, 10, and/or components thereof.

In step 305, one or more simulated optical inputs for the generation of one or more sets of simulated light transmission data as light travels through a physical model of tissue may be selected, received, and/or configured. The physical model of tissue may comprise one or more layers that have the same or different optical properties. The physical model may be made from for example, gels, aqueous solutions, and lipids. Exemplary optical inputs include, but are not limited to, light wavelength, intensity, modulation of the light (e.g., a duration of successive light pulses), and/or a range of wavelengths. In many cases, the optical inputs will be for the generation of infra-red and/or near infra-red light.

Next, in step 310, a plurality of sets of detected electronic signals corresponding to light generated using the optical inputs of step 305 that has passed through the physical model and been detected by a photodetector such as detector 160 may be received from the photodetector. In some cases, the detected electronic signals may correspond to light being transmitted through the physical model for a period of time (e.g., 15, 30, or 60 seconds; 1, 5, or 10 minutes). A result of execution of step 310 may be the generation of a set of simulated light transmission data. Step 310 may be executed a plurality (e.g., 50,000; 100,000; 500,000; 1,000,000; 5,000,000) of times thereby generating a plurality of sets of simulated light transmission data. The plurality of sets of detected electronic signals may then be stored in a database (step 315) like database 15 and/or 170.

In step 320, an oximetry value for each set of detected electronic signals may be determined and/or received. The oximetry value may be, for example, a maternal hemoglobin oxygen saturation level, a maternal tissue oxygenation level, a fetal hemoglobin oxygen saturation level, and/or a fetal tissue oxygenation level. When the oximetry values are hemoglobin oxygen saturation levels, the oximetry values may be determined via, for example, the Beer Lambert Law or a modified version of the Beer Lambert Law as explained above using Equations 1 and 2. When the oximetry values are tissue oxygen saturation levels, the oximetry values may be determined via, for example, diffuse optical tomography (DOT) or another tissue oxygen saturation determination technique. Following step 320, the oximetry values and/or correlations between each set of detected electronic signals (which may also be referred to herein as simulated light transmission data) and its respective oximetry value may be stored in a database (step 325) like database 15 and/or 170.

FIG. 3B is a flowchart illustrating an exemplary process 301 for developing and/or training a physiological indicator model that may be used to, for example, recognize patterns within one or more sets of values for physiological indicators and/or generate evaluations, diagnoses, and/or predictions based one or more patterns recognized within the within one or more sets of values for physiological indicators. The physiological indicator model may be incorporated into, for example, the in vivo fetal oximetry models disclosed herein, to for example, generate an augmented in vivo fetal oximetry model, which may be used to, for example, determine one or more fetal health indicators as disclosed herein. In some embodiments, the physiological indicator models generated via execution of process 301 may be incorporated into a fetal oximetry model that may be generated via execution of one or more of the processes disclosed herein. Process 301 may be executed by, for example, one or more systems and/or system component disclosed herein.

In step 355, a plurality of sets of values for one or more physiological indicators for a pregnant mammal and/or fetus and corresponding evaluations of and/or diagnoses for each of the plurality of sets of values for one or more physiological indicators may be received. Exemplary physiological indicators include but are not limited to fetal and/or maternal, photoplethysmogram (PPG) signals or values, heart rate, pulse, respiratory rate, uterine contraction information (e.g., duration and/or frequency), duration of labor and delivery of a fetus, gestational age of the fetus, chronological age of the pregnant mammal, maternal blood pressure, maternal skin tone, and hemoglobin and/or tissue oxygenation levels. The sets of values for the physiological indicators may include values that are measured at a point in time (e.g., a single value) and/or over a period of time (e.g., 30 seconds, 60 seconds, 5 minutes, 30 minutes, etc.). At times, values included in the one or more sets of values for the physiological indicators may be, for example, raw data, filtered data, time averaged data, and/or trend data. Exemplary evaluations and/or diagnoses that may correspond to the one or more of the plurality of sets of values for one or more physiological indicators include, but are not limited to, fetal distress, maternal distress, fetal hypoxia, binary fetal hypoxia, fetal hypoxemia, fetal non-hypoxia, fetal non-hypoxemia, and/or preeclampsia.

The plurality of sets of values of physiological indicators may be divided into a training set and a testing set (step 360) and machine learning inputs may be selected and/or set (step 365). Exemplary inputs include, but are not limited to, equipment characteristics, background noise characteristics, maternal geometrical characteristics, maternal physiological characteristics, fetal geometrical characteristics, fetal physiological characteristics and/or maternal oximetry values (e.g., SpO2). Input features may be normalized to standard mean and/or variance values, such as zero mean and unit variance, and, in some instances, may be combined into composite features that are then input into the machine learning architecture. In some cases, the machine learning architecture disclosed herein may be a deep learning network architecture that may include convolutional nets and engineered feature layers. Additionally, or alternatively, the machine learning architecture may be a neural network, an artificial neural network, a Bayesian network, and/or software or hardware that utilizes artificial intelligence. In some embodiments, execution of step 365 may include down sampling and/or activating one or convolutional layers of the machine learning architecture and/or a model generated by the machine learning architecture. In some cases, execution of step 365 may also include adding one or more engineered features, bias, and/or classifier layers to the machine learning architecture and/or a model. Additionally, or alternatively, physiological indicator models generated via execution of process 301 may include tree-based models or ensembles of layered and/or tree-based models. Additionally, or alternatively, physiological indicator models generated via execution of process 301 may incorporate K-fold cross-validation to, for example, generate the expected error, receiver operating characteristic (ROC), and/or area under the curve (AUC) values for the model.

At times the machine learning inputs may be, for example, one or more pattern-recognition algorithms. In some embodiments, execution of step 365 may include communication of the machine learning inputs and/or machine learning architecture to, for example, a machine learning computer platform and/or neural network such as a machine learning platform resident on/within cloud computing platform 11.

In step 370, a first version of a physiological indicator model may be trained to recognized one or more patterns within the plurality of sets of values of physiological indicators included in the training set that may, or may not be, associated with the evaluations and/or diagnoses of the respective plurality of sets of values of physiological indicators. The first version of the physiological indicator model may then be stored (step 375) and tested using the testing set of values of physiological indicators (step 380). Results of the testing may be evaluated (step 385) and used to revise and/or update the first version of the physiological indicator model thereby generating a second version of the physiological indicator model. In some embodiments, the physiological indicator model may be a formula or portion of a formula that may be an input variable, or subroutine, of a fetal oximetry model such as the simulated augmented fetal oximetry models and/or augmented in vivo fetal oximetry models disclosed herein. Additionally, or alternatively, the physiological indicator model may include one or more adjustments (or instructions for adjustments) to be made to a simulated augmented fetal oximetry model and/or augmented in vivo fetal oximetry model in order to, for example, augment, or personalize, the simulated augmented fetal oximetry model and/or augmented in vivo fetal oximetry models to one or more physiological inputs.

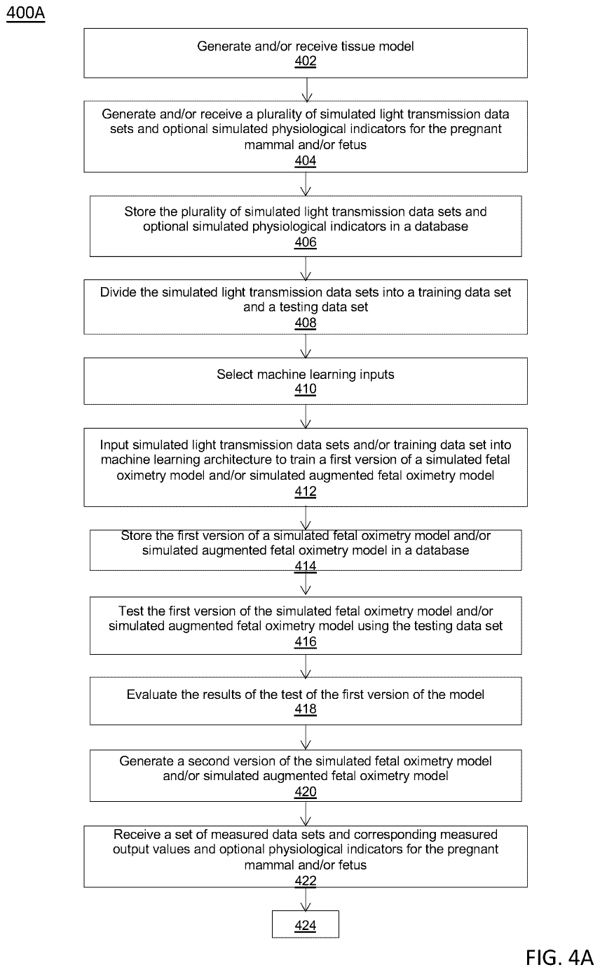

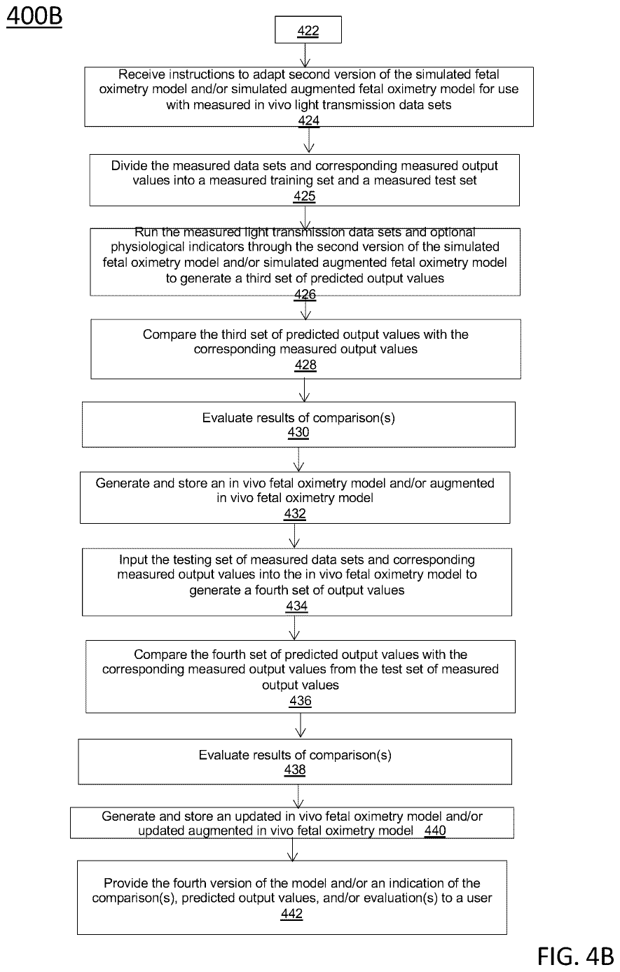

FIGS. 4A and 4B provide a flowchart (over two pages) showing an exemplary process 400 for developing a model to accurately calculate oximetry values for a target tissue within a body, such as a fetus in-utero. Process 400 may be executed by, for example, system 100, 10, and/or components thereof.

Initially, in step 402, a tissue model may be received (e.g., following execution of process 200 and/or 300) and/or generated using, for example, a process similar to process 200 and/or 300. The tissue model may be a two and/or three-dimensional model of a portion of an animal (e.g., human) body with one or more layers of tissue. The modeled layer(s) of tissue may have different optical properties. An exemplary tissue model is provided by model 900 shown in FIG. 9, as discussed above. For the purposes of discussion, a layer of the tissue model may correspond to a model of abdominal tissue for a pregnant mammal and another layer may correspond to a model of fetal tissue but, it is noted that the models of other parts of the body that have two or more layers may also be received and/or generated in step 402. In some cases, execution of step 402 may also include receipt and/or selection of parameters or rules for the model such as the parameters showing in table 1000 of FIG. 10, discussed above.

In some embodiments, a PPG modulated signal may be integrated into one or more of the models generated in step 402 to simulate cardiac cycles for the pregnant mammal and/or fetus. The PPG modulated signal may have, for example, a variable 1% to 2% change in systolic blood volume for the pregnant mammal and fetus. FIG. 11 provides a graph 1100 that plots a simulated fetal and maternal PPG signals over time in seconds, wherein the PPG signal for the mother/pregnant mammal is shown in blue and the PPG signal for the fetus is shown in red. In some embodiments, noise and/or a confounding factor may be added to the PPG signal for the fetus and/or pregnant mammal as part of, for example, perturbation analysis using the models.

In step 404, a plurality (e.g., 500,000; 1,000,000; 5,000,000) of simulated light transmission data sets that simulate light traveling, over a period of time (e.g., 10 s, 30 s, 60 s, 5 minutes, etc.), through the models received and/or generated in step 402 and being detected by a detector like detector 160 may be generated and/or received. In some embodiments, execution of step 404 may include generation of one or more time series waveforms with variable fetal (100 to 240 BPM) and/or maternal (50 to 12 BPM) heartrates, amplitudes, and phases between them. Individual parameters used to generate each of the simulated light transmission data sets may be selected randomly, pseudo randomly, and/or systematically according to, for example, a physiologically appropriate distribution (e.g., likelihood of occurrence within a population) assigned to each parameter.

In some embodiments, execution of step 404 may include running a plurality (e.g., 50-500) of experiments and/or simulations with different inputs (e.g., fetal and maternal cross correlation with heartbeats, DC level, maternal SpO2, normalization ratios, fetal depth, and/or maternal optical scattering properties), different machine learning architectures. In some cases, different classifiers and/or loss functions may generate a large number (e.g., 2-5 million) of data sets from which fetal oximetry values (e.g., fetal SpO2, fetal tissue oxygen saturation, etc.) may be calculated. Additionally, or alternatively, execution of step 404 may include running a plurality (e.g., 50-500) of experiments and/or simulations with different inputs that pertain to features of equipment (e.g., detector sensitivity, lag times, light source characteristics, errors or noise that may be introduced into a signal when particular equipment is used, etc.) that may be used when taking in vivo light transmission and/or fetal oximetry measurements.

In some embodiments, execution of step 404 may include generating simulated light transmission data sets where the light transmission data is “received” from a plurality (e.g., 2, 4, 6, or 8) of different detectors and/or is transmitted by a plurality (e.g., 2, 4, 6, or 8) of sources. Additionally, or alternatively, the simulated detector signals may correspond to light of different wavelengths and/or from different types of light sources. Additionally, or alternatively, execution of step 404 may include generating desired features of the simulated light transmission data sets such as, for example, correlation amplitudes, DC levels, time of flight for photons detected upon emission from a pregnant mammal's abdomen, and/or fast Fourier transforms (FFTs). In some embodiments, execution of step 404 may include calculating one or more correlation amplitudes for the simulated light transmission data sets using, for example, time series data.

On some occasions, the simulated light transmission data sets generated in step 404 may include calibration formulas that relate, for example, a ratio of ratios (R) and/or an optical density of tissue with fetal oximetry values. On some occasions, a plurality (e.g., 100-100,000) calibration formulas may be generated via execution of step 404 using by incorporating various factors and/or inputs into the model used to generate the simulated light transmission sets. For example, generation of one or more of the pluralities of calibration formulas may include simulating light's transmission through tissue models with varying optical properties such as a scattering coefficient and/or absorption coefficient for the tissue model, varying fetal depth, varying wavelengths of light transmitted through the tissue model and/or varying maternal hemoglobin oxygen saturation levels.

In some embodiments, simulated values for one or more physiological indicators and/or measurements for the pregnant mammal and/or fetus may be received and/or generated in step 404. Exemplary values for simulated physiological indicators include, but are not limited to, maternal and/or fetal heart rate, maternal age, fetal gestational period, maternal and/or fetal ECG data, maternal and/or fetal temperature, maternal and/or fetal pulse, uterine contraction information, maternal respiratory information, maternal oximetry information, and/or maternal and/or fetal ultrasound data. At times, correlations between values for the received and/or generated simulated physiological indicators and evaluations of these values with regard to implications for the health of the fetus and/or pregnant mammal may also be received and/or determined in step 404.

The received and/or generated simulated light transmission data sets and, when appropriate, simulated physiological indicators may then be stored in a database like database 15 and/or 170 (step 406). Optionally, in step 408, the simulated light transmission data sets may then be divided into a training set (e.g., 60%, 70%, or 80% of the data sets) and a testing set (e.g., 40%, 30%, or 20% of the data sets).

Optionally, in step 410, inputs to the machine learning architecture and/or software program for determining fetal oximetry values may be selected. Exemplary inputs include, but are not limited to, fetal depth, fetal heart rate, maternal heart rate, equipment characteristics, background noise characteristics, maternal geometrical characteristics, maternal physiological characteristics, fetal geometrical characteristics, fetal physiological characteristics, one or more physiological indicators, an evaluation of a physiological indicator, and/or maternal oximetry values (e.g., SpO2). In some embodiments, one or more inputs may be received from a component of system 100 such as ECG 175, Doppler/ultrasound probe 135, pulse oximetry probe 130, NIRS adult hemoglobin probe 125, and/or ventilator/ventilatory signal device. Input features may be normalized to standard mean and/or variance values, such as zero mean and unit variance, and, in some instances, may be combined into composite features that are then input into the machine learning architecture. In some cases, the machine learning architecture disclosed herein may be a deep learning network architecture that may include convolutional nets and engineered feature layers. Additionally, or alternatively, the machine learning architecture may be a neural network, an artificial neural network, a Bayesian network, and/or software or hardware that utilizes artificial intelligence. In some embodiments, execution of step 410 may include down sampling and/or activating one or convolutional layers of the machine learning architecture and/or a model (e.g., a simulated fetal oximetry model and/or simulated augmented fetal oximetry model) generated by the machine learning architecture. In some cases, execution of step 410 may also include adding one or more engineered features, bias, and/or classifier layers to the machine learning architecture and/or a model (e.g., a simulated fetal oximetry model and/or simulated augmented fetal oximetry model) generated by the machine learning architecture. Additionally, or alternatively, models (e.g., simulated fetal oximetry model and/or simulated augmented fetal oximetry models) generated by process 400 may include tree-based models or ensembles of layered and/or tree-based models. Additionally, or alternatively, models (e.g., simulated fetal oximetry model and/or simulated augmented fetal oximetry models) generated by process 400 may incorporate K-fold cross-validation to, for example, generate the expected error, receiver operating characteristic (ROC), and/or area under the curve (AUC) values for the model.

In some embodiments, execution of step 410 may include selection of one or more types of outputs that may be incorporated into the machine learning architecture. Exemplary outputs include predicted fetal oximetry (e.g., SpO2 and/or fetal tissue oxygen saturation) values and a binary fetal hypoxia, fetal hypoxemia, fetal non-hypoxia, and/or fetal non-hypoxemia (e.g., fetal SpO2 above/below 30%) indication. Additionally, or alternatively, execution of step 410 may include incorporation of a physiological indicator model such as a physiological indicator model generated via execution of step 301 into the machine learning architecture.

In step 412, the simulated light transmission data sets and/or training data set (when step 408 is executed) may be input into the machine learning architecture to generate and/or train a first version of a simulated fetal oximetry model and/or a simulated augmented fetal oximetry model (when, for example, physiological indicators and/or an evaluation of physiological indicators are received in step 404) that may be configured to, for example, to predict a first set of outputs (e.g., fetal SpO2 values, fetal tissue oxygen saturation, and/or fetal hypoxemia or non-hypoxemia determinations). The first version of the simulated fetal oximetry model and/or simulated augmented fetal oximetry model may include a plurality of layers and/or functions and, in some cases, may include one or more small layered network(s), sub-networks, and/or a Support Vector Machine. In some embodiments, execution of step 412 may include communication of the machine learning inputs and/or machine learning architecture to, for example, a machine learning computer platform and/or neural network such as a machine learning platform resident on/within cloud computing platform 11. In some cases, the augmented simulated fetal oximetry model may include, or otherwise incorporate, the physiological indicator model. In step 414, the first version of the simulated fetal oximetry model and/or simulated augmented fetal oximetry model may be stored in a database such as database 15 and/or 170.

Optionally, in step 416, the first version of the simulated fetal oximetry model and/or simulated augmented fetal oximetry model and/or first set of outputs may be tested using, for example, the testing data set from step 408. The results of the testing may then be evaluated (step 418) and used to modify the first version of the simulated fetal oximetry model and/or simulated augmented fetal oximetry model thereby generating a second version of the simulated fetal oximetry model and/or simulated augmented fetal oximetry model (step 420) via, for example, training and/or tuning the first version of the simulated fetal oximetry algorithm using the machine learning architecture. The second version of the simulated fetal oximetry model and/or simulated augmented fetal oximetry model may be used to predict a second set of outputs. In some embodiments, the second version of the simulated fetal oximetry model and/or simulated augmented fetal oximetry model may be similar, or identical to, the first version of the fetal oximetry model. At times, the evaluation of step 418 may be responsive to one or more physiological indicators and/or an evaluation thereof. Additionally, or alternatively, execution of step 418 may include comparing a result from a simulated fetal oximetry model and a simulated augmented fetal oximetry model to, for example, determine differences therebetween and/or how input of the physiological indicators and/or an evaluation of the physiological indicators into the simulated augmented fetal oximetry model impacted a result of the testing and/or outputs of the augmented simulated fetal oximetry model when compared with the simulated augmented fetal oximetry model. A result of this evaluation may assist with determining how important inclusion of a particular physiological indicator is to yielding an accurate output from the model.

Then, in step 422, a set of measured, or actual, in vivo light transmission data sets and corresponding output data (e.g., fetal oximetry values such as fetal SpO2 and/or fetal tissue oxygenation saturation) may be received. The in vivo light transmission data sets may be received from a fetal oximetry probe such as fetal oximetry probe 115 and each corresponding output data/oximetry value may be calculated using a corresponding in vivo light transmission data set received in step 422. In one embodiment, the set of measured in vivo light transmission data sets and corresponding measured output data may include 200-10,000 datasets/output values. In the case of a pregnant human, the measured output data may be light transmission data sets taken over an interval of time (e.g., 30 or 60 seconds) and the measured, or actual, output values may be measured in vivo fetal oximetry values corresponding, in time, to when the light transmission data sets were measured. The In this example, measured in vivo fetal oximetry values may be within the range of, for example, of 10-70%. In some cases, the set of set of measured, or actual, output data may be converted into a format compatible with the predicted outputs so that a valid comparison between them may be made.

Optionally, values and/or measurements one or more physiological indicators (e.g., measurements and/or parameters) for a pregnant mammal and/or fetus associated with the received measured data sets may be received in step 422 from, for example, one or more components of system 100 and/or a fetal oximetry probe like fetal oximetry probe 115 that may be transabdominal and/or internally placed within the pregnant mammal's vagina and/or uterus. Exemplary physiological indicators include, but are not limited to, maternal and/or fetal heart rate, maternal age, fetal gestational period, maternal and/or fetal ECG data, maternal and/or fetal temperature, maternal and/or fetal pulse, uterine contraction information, maternal respiratory information, maternal oximetry information, and/or maternal and/or fetal ultrasound data. On some occasions, physiological indicators received in step 422 may include and/or be correlated to evaluations or assessments of the physiological indicator(s) and execution of step 422 may include receipt of a physiological indicator for the pregnant mammal and/or fetus and querying a database for one or more evaluations or assessments that may be associated with the received physiological indicator. For example, a measured fetal heart rate may be received in step 422 and a database may be queried for one or more correlated evaluations of the measured fetal heart rate. The fetal heart rate and the evaluation may then be input into the augmented in vivo fetal oximetry value to determine an oximetry value for the fetus as well as other indicators of fetal distress (e.g., fetal heart rate is too low or too high).

In step 424, instructions to adapt the first or second (when steps 416-420 are performed) version of the simulated fetal oximetry model and/or simulated augmented fetal oximetry model for use in the generation of a first version of an in vivo fetal oximetry model and/or augmented in vivo fetal oximetry model. The first version of the in vivo fetal oximetry model and/or augmented in vivo fetal oximetry model may be generated by training, for example, the first/second version of the simulated fetal oximetry model and/or simulated augmented fetal oximetry model using a plurality of measured in vivo light transmission data sets and corresponding measured in vivo fetal oximetry values.

Exemplary instructions received in step 424 include instructions to train, or update, only certain portions (e.g., layers, functions, networks, and/or sub-networks) of the first/second version of the simulated fetal oximetry model and/or simulated augmented fetal oximetry model and fix, or hold constant, other portions of the fetal first/second version of the simulated fetal oximetry model and/or simulated augmented fetal oximetry model as needed. Typically, the initial input layer or layers of the network would be fixed to preserve the features found in the simulations. Additionally, or alternatively, portions of the first/second version of the simulated fetal oximetry model and/or simulated augmented fetal oximetry model that may remain fixed include portions of the first/second version of the simulated fetal oximetry model and/or simulated augmented fetal oximetry model that are generally applicable to the in vivo fetal oximetry model and/or augmented in vivo fetal oximetry model such as, for example, layers pertaining to calibration factors, calibration curves, calibration formulas, maternal physiology and/or geometry, fetal physiology and/or geometry, and/or equipment parameters.

Optionally, in step 425, the measured in vivo light transmission data sets and corresponding output values (e.g., fetal oximetry values) may be divided into a measured training set and a measured testing set. In step 426, the in vivo light transmission data sets and corresponding output data (e.g., oximetry values) and/or the training set of in vivo light transmission data sets and corresponding output data (when step 424 is executed) may be input into an adapted (according to the instructions of step 424) version of the first/second version of the simulated fetal oximetry model and/or simulated augmented fetal oximetry model so that one or more portions (e.g., layers or functions) of the first/second fetal oximetry model may be tuned or updated using the in vivo light transmission data and corresponding output values thereby generating an in vivo fetal oximetry model and/or augmented in vivo fetal oximetry model and, optionally, a third set of predicted output values generated by the in vivo fetal oximetry model and/or augmented in vivo fetal oximetry model.

In step 428, the third set of predicted output values may be compared with the corresponding measured output values to determine differences between them (step 428). Results of the comparison may then be evaluated (step 430) and used to update the in vivo fetal oximetry model and/or augmented in vivo fetal oximetry model (step 432). Execution of step 432 may also include storing the updated in vivo fetal oximetry model and/or augmented in vivo fetal oximetry model in a database such as the databases disclosed herein. In some cases, the augmented in vivo fetal oximetry model may include, or otherwise incorporate, the physiological indicator model.

Optionally, when step 425 is performed, the testing set of measured light transmission data and corresponding output values may then be run through the in vivo fetal oximetry model and/or augmented in vivo fetal oximetry model to generate a fourth set of predicted output values (step 434). The fourth set of predicted output values may then be compared with the corresponding measured output values from the testing set of output values to determine differences between them (step 436). Results of the comparison may then be evaluated (step 438) and used to generate an updated in vivo fetal oximetry model and/or augmented in vivo fetal oximetry model to predict output values (step 440) using the machine learning architecture. The updated in vivo fetal oximetry model and/or augmented in vivo fetal oximetry model may also be stored in step 440. Then, the in vivo fetal oximetry model and/or augmented in vivo fetal oximetry model and/or an indication of the comparison(s), evaluation(s), and/or predicted output values may be provided to the user (step 442).

In some embodiments, process 400 and/or portions thereof may be repeated on a periodic, as needed, and/or continuous basis to, for example, improve the accuracy of the predictions the model yields, perform perturbation analysis, and/or perform sensitivity analysis. When step 434 is not performed, process 400 may end at step 432.

In some embodiments, execution of steps 428 and/or 430 may include comparing the third set of predicted output values to their respective measured output values for both the simulated fetal oximetry model and the simulated augmented fetal oximetry model to, for example, determine differences therebetween and/or how input of the physiological indicators and/or an evaluation of the physiological indicators into the simulated augmented fetal oximetry model impacted the third set of predicted output values and/or an evaluation of the third set of predicted output values to, for example, determine whether the third set of predicted output values are more accurate for the simulated fetal oximetry model or the simulated augmented fetal oximetry model. At times, execution of steps 436 and/or 438 may include similar evaluations and/or determinations. A result of these evaluations and/or determinations may assist with determining sensitivity of the fetal oximetry models to inclusion of a particular physiological indicator. Additionally, or alternatively, a result of these evaluations and/or determinations may assist with determining how important inclusion of a particular physiological indicator is to yielding an accurate output from the model. Additionally, or alternatively, a result of these evaluations and/or determinations may assist clinicians in deciding which physiological indicators and/or which combinations of physiological indicators to add to an augmented in vivo fetal oximetry model.

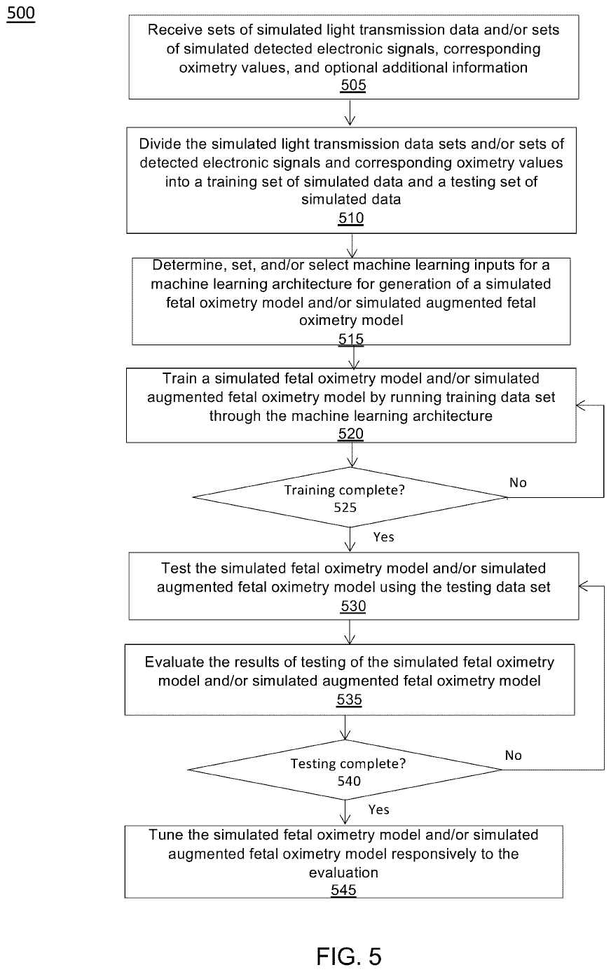

FIG. 5 is a flowchart illustrating an exemplary process 500 for the generation of a simulated fetal oximetry model and/or simulated augmented fetal oximetry model and/or a tuned simulated fetal oximetry model and/or simulated augmented fetal oximetry model. Process 500 may be performed by, for example, any of the systems or system components disclosed herein and may use data, determinations, and/or models generated and/or used by any of the processes disclosed herein.

Initially, a plurality (e.g., 10,000-10 million) of sets of simulated light transmission data and corresponding oximetry values for each set of simulated light transmission data may be received by a processor or network of processors such as cloud computing platform 11 (step 505). Each set of the simulated light transmission data may have been generated by simulating a transmission of light of one more wavelengths and/or intensities through a model of animal tissue that may have been generated and/or received via, for example, execution of process 200 and/or 300. The oximetry values corresponding to each set of simulated light transmission data may have been generated via, for example, execution of process 200 and/or 300 and/or may be calculated as part of execution of step 505 using the simulated light transmission data. In some embodiments, the model of animal tissue may include at least two layers of animal tissue, one of which is fetal tissue (e.g., skin, bone, brain, blood, etc.). Optionally, in step 505, additional information regarding one or more of sets of simulated light transmission data and/or oximetry values may be received. The additional information may pertain to, for example, one or more of the following: fetal depth, source/detector separation distance, a thickness of maternal tissue, a type of maternal tissue, maternal and/or fetal skin color and/or melanin content, a thickness of fetal tissue, a type of fetal tissue, a type of light used, an intensity of light used, a light scattering property of layer of tissue in the model, a light absorption property of layer of tissue in the model, a fetal age, a calibration formula, a calibration formula specific to a particular patient characteristic and/or patient, and/or calibration factor(s) associated with equipment used to obtain the simulated light transmission data, environmental conditions when the simulated light transmission data is collected. Additionally, or alternatively, the additional information received in step 505 may be simulated and/or previously measured values for physiological indicators.

Optionally, the plurality of sets of simulated light transmission data and corresponding fetal oximetry values may be divided into a training set of simulated data and a test set of simulated data (step 510). The plurality of sets of simulated light transmission data and corresponding fetal oximetry values may be divided along any appropriate ratio including, for example, 90:10 training/testing; 80:20 training/testing; or 70:30 training/testing. In some embodiments, execution of step 510 may be similar to execution of step 408.

In step 515, machine learning inputs for the generation of a simulated fetal oximetry model and/or simulated augmented fetal oximetry model (i.e., a simulated fetal oximetry model augmented and/or configured to incorporate values for physiological indicator(s) and/or evaluations of the physiological indicators) may be determined, set, and/or selected for input into a machine learning program and/or architecture such as herein described. In some embodiments, execution of step 515 may resemble execution of step 410. Then, in step 520, a simulated fetal oximetry model and/or simulated augmented fetal oximetry model may be trained using all or most of the data (in all or most combinations) received in step 505 and/or the training set of data of step 510 when step 510 is executed. Step 520 may be executed via, for example, inputting the simulated light transmission data, simulated detected electronic signals, corresponding oximetry values and/or addition information and/or a training set thereof (when step 510 is executed) into the machine learning architecture once it is set up with the machine learning inputs of step 515. The simulated fetal oximetry model and/or simulated augmented fetal oximetry model may be configured to receive a plurality of sets of simulated light transmission data included in the training set of simulated data and determine an oximetry value a fetus for each set of simulated light transmission data included in the training set of simulated data. This determined oximetry value may then be compared with the corresponding oximetry value received in step 505 to determine any differences therebetween. Results of this comparison may be used to iteratively update/train the simulated fetal oximetry model and/or simulated augmented fetal oximetry model during execution of step 520. Training of the simulated fetal oximetry model and/or simulated augmented fetal oximetry model may be complete (step 525) when, for example, a number or proportion (e.g., 60-99%) of the oximetry values calculated by the simulated fetal oximetry model and/or simulated augmented fetal oximetry model using one or more simulated light transmission data sets received in step 505 are sufficiently close to (e.g., within a standard of deviation, within 0.5 standards of deviation, within 0.1 standards of deviation, and/or within 60-99% of the associated oximetry value) of the oximetry values associated each of the respective simulated light transmission data sets. When the training of the simulated fetal oximetry model and/or simulated augmented fetal oximetry model is not complete (step 525), step 520 may be repeated. When step 510 is not executed, process 500 may end following a determination that the training of the simulated fetal oximetry model and/or simulated augmented fetal oximetry model in step 525 is complete.

In some embodiments, the simulated fetal oximetry model and/or simulated augmented fetal oximetry model includes a plurality of layers, factors, calibrations, calibration formulas, and/or functions (referred to herein collectively as “layers”) that are used to calculate oximetry values using the simulated light transmission data. Layers may include functions that account for and/or factor in, for example, fetal depth, source/detector separation distance, a thickness of maternal tissue, a type of maternal tissue, maternal and/or fetal skin color and/or melanin content, a thickness of fetal tissue, a type of fetal tissue, a wavelength of light used, an intensity of light used, a fetal age, calibration formulas, and/or calibration factor(s) associated with equipment that may be used in clinical applications to obtain in vivo measurements of light transmission data, environmental conditions that may be present during clinical applications when in vivo measurements of light transmission data is collected. In some cases, the augmented in vivo fetal oximetry model may include, or otherwise incorporate, the physiological indicator model.

When the training of the simulated fetal oximetry model and/or simulated augmented fetal oximetry model is complete (step 525), the simulated fetal oximetry model and/or simulated augmented fetal oximetry model may be tested with the testing set of simulated data (step 530). In some embodiments, execution of step 530 may be similar to execution of step 416. Results of the testing of the simulated fetal oximetry model and/or simulated augmented fetal oximetry model may then be evaluated (step 535) to, for example, determine how accurately the simulated fetal oximetry model and/or simulated augmented fetal oximetry model calculated oximetry values. In some cases, the testing of step 530 may be iterative. When the testing of the simulated fetal oximetry model and/or simulated augmented fetal oximetry model is complete (step 540), the simulated fetal oximetry model and/or simulated augmented fetal oximetry model may be tuned responsively to one or more results of the testing and/or evaluation of the tests (step 545) thereby generating a tuned simulated fetal oximetry model and/or simulated augmented fetal oximetry model and process 500 may end. When the testing of the simulated fetal oximetry model and/or simulated augmented fetal oximetry model is not complete (step 540), process 500 may proceed to step 530.

In some embodiments, execution of steps 530 and/or 535 may include comparing test results and/or an evaluation of the test results for the simulated fetal oximetry model and the simulated augmented fetal oximetry model to, for example, determine differences therebetween to, for example, determine a sensitivity of the fetal oximetry models to inclusion of a particular physiological indicator.

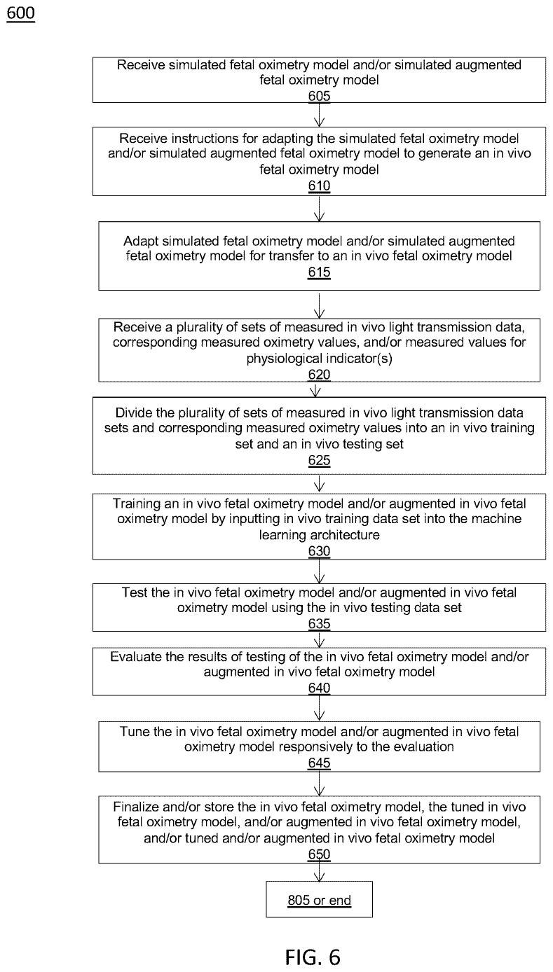

FIG. 6 is a flowchart illustrating an exemplary process 600 for the generation of an in vivo fetal oximetry model and/or augmented in vivo fetal oximetry model and/or a tuned in vivo fetal oximetry model and/or augmented in vivo fetal oximetry model. Process 600 may be performed by, for example, any of the systems or system components disclosed herein and may use data, measurements, evaluations, determinations, and/or models generated and/or used by any of the processes disclosed herein. In some embodiments, process 600 may be performed subsequently to performance of process 400 and/or 500 and, on occasion, may be executed by the same systems and/or processors.

In step 605, a simulated fetal oximetry model and/or simulated augmented fetal oximetry model, such as the simulated fetal oximetry model and/or simulated augmented fetal oximetry model generated by process 400 and/or a tuned simulated fetal oximetry model and/or simulated augmented fetal oximetry model, such as the tuned simulated fetal oximetry model and/or simulated augmented fetal oximetry model generated by process 500, may be received by, for example, a processor or network of processors such as cloud computing platform 11. Additionally, or alternatively, when, for example, steps 530-545 of process 500 are executed, a tuned simulated fetal oximetry model and/or simulated augmented fetal oximetry model may be received in step 605. For ease of discussion, the simulated fetal oximetry model and/or the simulated augmented fetal oximetry model (of, for example, step 525) and the tuned simulated fetal oximetry model and/or simulated augmented fetal oximetry model (of, for example, step 545) may be referred to herein as the “simulated fetal oximetry model and/or simulated augmented fetal oximetry model.”

Instructions to adapt the simulated fetal oximetry model and/or simulated augmented fetal oximetry model for transfer to an in vivo fetal oximetry model and/or augmented in vivo fetal oximetry model may then be received (step 610). In some cases, the instructions to adapt the simulated fetal oximetry model and/or simulated augmented fetal oximetry model for transfer to an in vivo fetal oximetry model and/or augmented in vivo fetal oximetry model may include instructions to fix one or more layers, or functions, of the simulated fetal oximetry model and/or simulated augmented fetal oximetry model that may be generally applicable to the in vivo fetal oximetry model and/or augmented in vivo fetal oximetry model. Exemplary layers and/or functions of the tuned simulated fetal oximetry model and/or simulated augmented fetal oximetry model that may be fixed include, but are not limited to, how one or more of a source/detector distance, a wavelength of light, a fetal depth, maternal skin color, fetal skin color, maternal tissue composition, fetal tissue composition and/or a calibration factor impact (e.g., weights in the model), an oximetry calculation. In some cases, the augmented simulated fetal oximetry model may include, or otherwise incorporate, the physiological indicator model.

Then, the tuned simulated fetal oximetry model and/or simulated augmented fetal oximetry model may be adapted for transfer to an in vivo fetal oximetry model and/or augmented in vivo fetal oximetry model responsively to the instructions (step 615). In some cases, the adapting of step 615 may include determining, setting, and/or selecting one or more machine learning inputs for a machine learning architecture for the generation of an in vivo fetal oximetry model and/or augmented in vivo fetal oximetry model. Additionally, or alternatively, the adapting of step 615 may include fixing one or more layers, or functions, of the tuned simulated fetal oximetry model and/or simulated augmented fetal oximetry model so that it remains fixed during the in vivo fetal oximetry model and/or augmented in vivo fetal oximetry model training process (step 630, which is discussed below).

In step 620, a plurality (e.g., 1,000-10 million) of sets of in vivo light transmission data and corresponding fetal oximetry values for each set of in vivo light transmission may be received. The plurality of sets of in vivo light transmission data may be received from, for example, a fetal oximetry probe like fetal oximetry probe 115 and the corresponding fetal oximetry values may be calculated using, for example, Equations 1 and 2 as discussed herein. Optionally, one or more measured values for one or more physiological indicators (e.g., heart rate, ECG values, changes in heart rate over time, etc.) of the pregnant mammal and/or fetus may also be received in step 620.

Optionally, in step 625, the plurality of sets of in vivo light transmission data and corresponding fetal oximetry values and optional values for one or more physiological indicators may then be divided into a training set of in vivo data and a test set of in vivo data. This division may occur in any appropriate proportion and/or ratio including, for example, 90:10 training/testing; 80:20 training/testing; or 70:30 training/testing. In some embodiments, execution of step 625 may have one or more similarities with execution of step 408 and/or 510.

In step 630, an in vivo fetal oximetry model and/or augmented in vivo fetal oximetry model may be trained using the training set of in vivo data and the adapted simulated fetal oximetry model and/or simulated augmented fetal oximetry model of step 615. Step 630 may be executed via, for example, inputting the training set of in vivo data into the machine learning architecture once it is set up with the adapted simulated fetal oximetry model and/or simulated augmented fetal oximetry model of step 615. In some embodiments, the in vivo fetal oximetry model and/or augmented in vivo fetal oximetry model may be configured to receive a plurality of sets of in vivo light transmission data included in the plurality of sets of measured in vivo data and/or training set of in vivo data and determine an oximetry value of a fetus for each respective set of in vivo light transmission data and/or determine one or more indications of fetal health, such as “not in distress,” “in distress,” “declining, or “improving” based on, for example, the oximetry values and one or more physiological indicator values (e.g., low fetal heart rate in combination with low fetal oximetry values).

The determined oximetry value of step 630 may then be compared with the oximetry value associated with the in vivo light transmission data to determine any differences therebetween. These differences may be used to, for example, iteratively update/train the in vivo fetal oximetry model and/or augmented in vivo fetal oximetry model during execution of step 630. Training of the in vivo fetal oximetry model and/or augmented in vivo fetal oximetry model may be complete when, for example, a number or proportion (e.g., 60-99%) of the oximetry values calculated by the in vivo fetal oximetry model and/or augmented in vivo fetal oximetry model using one or more in vivo light transmission data sets received in step 620 are sufficiently close to (e.g., within a standard of deviation, within 0.5 standards of deviation, within 0.1 standards of deviation, and/or within 60-99% of the associated oximetry value) to the oximetry values associated with each of the respective in vivo transmitted light data sets. When the training of the in vivo fetal oximetry model and/or augmented in vivo fetal oximetry model is not complete, step 630 may be repeated and/or may continue to be executed. Additionally, or alternatively, the determined fetal health of step 630 may then be compared with, for example, the oximetry value associated with the in vivo light transmission data and a value for the physiological indicator to determine any differences therebetween.

In some embodiments, the in vivo fetal oximetry model and/or augmented in vivo fetal oximetry model includes a plurality of layers, factors, calibrations, and/or functions (referred to herein collectively as “layers”) that are used to calculate oximetry values using the in vivo light transmission data. Exemplary layers include functions that factor in, account for, and/or are associated with, for example, fetal depth, source/detector separation distance, a thickness of maternal tissue, a type of maternal tissue, maternal and/or fetal skin color and/or melanin content, a thickness of fetal tissue, a type of fetal tissue, a type of light used, an intensity of light used, a fetal age, and/or calibration factor(s) associated with equipment used to obtain the in vivo light transmission data, environmental conditions when the in vivo light transmission data is collected.

When the training of the in vivo fetal oximetry model and/or augmented in vivo fetal oximetry model is complete, process 600 may optionally proceed to step 650. Alternatively, and optionally, when the training of the in vivo fetal oximetry model and/or augmented in vivo fetal oximetry model is complete, the in vivo fetal oximetry model and/or augmented in vivo fetal oximetry model may be tested with the testing set of in vivo data (step 635). Optionally, results of the testing of the in vivo fetal oximetry model and/or augmented in vivo fetal oximetry model may then be evaluated (step 640) to, for example, determine how accurate the in vivo fetal oximetry model and/or augmented in vivo fetal oximetry model calculated oximetry values and/or fetal health determinations are. In some cases, the testing of step 635 may be iterative. When the testing of the in vivo fetal oximetry model and/or augmented in vivo fetal oximetry model is complete, the in vivo fetal oximetry model and/or augmented in vivo fetal oximetry model may be tuned and/or updated responsively to one or more results of the testing and/or evaluation of the tests (step 645) thereby generating a tuned in vivo fetal oximetry model and/or augmented in vivo fetal oximetry model. The tuned in vivo fetal oximetry model and/or augmented in vivo fetal oximetry model may then be finalized and/or stored and process 600 may end. At times, the tuned in vivo fetal oximetry model and/or tuned augmented in vivo fetal oximetry model may be referred to herein as the in vivo fetal oximetry model and/or augmented in vivo fetal oximetry model.

In some embodiments, execution of steps 635 and/or 640 may include comparing test results and/or an evaluation of the test results for the in vivo fetal oximetry model and the augmented in vivo fetal oximetry model to, for example, determine differences therebetween to, for example, determine a sensitivity of the fetal oximetry models to inclusion of a particular physiological indicator.

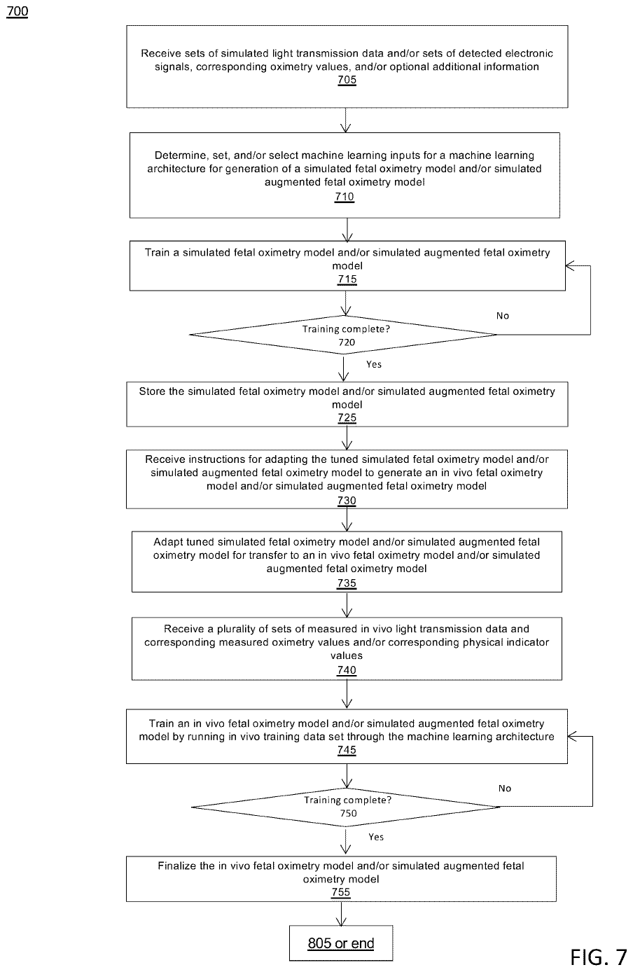

FIG. 7 is a flowchart illustrating an exemplary process 700 for the generation of an in vivo fetal oximetry model and/or augmented in vivo fetal oximetry model. Process 700 may be performed by, for example, any of the systems or system components disclosed herein and may use data, determinations, and/or models generated and/or used by any of the processes disclosed herein.

Initially, a plurality (e.g., 100,000-10 million) of sets of simulated light transmission data and corresponding oximetry values for each set of simulated light transmission data may be received by a processor or network of processors such as cloud computing platform 11 (step 705). Each set of the simulated light transmission data may have been generated by simulating a transmission of light of one more wavelengths and/or intensities through a model of animal tissue that may have been generated and/or received via, for example, execution of process 200 and/or 300. The simulated light transmission data sets may resemble those received in, for example, step 404. In some embodiments, the oximetry values corresponding to each set of simulated light transmission data may have been generated via, for example, execution of process 200 and/or 300 and/or may be calculated as part of execution of step 705 using the simulated light transmission data. In some embodiments, the model of animal tissue may include at least two layers of animal tissue, one of which is fetal tissue (e.g., skin, bone, brain, blood, etc.). On some occasions, execution of step 705 may resemble execution of step 505. Optionally, in step 705, additional information that may correspond to the simulated light transmission data, simulated detected electronic signals, and/or simulated oximetry values may also be received in step 705. The additional information received may be similar to the values for physiological indicators received in step(s) 505 and/or 620.

In step 710, machine learning inputs for the generation of a simulated fetal oximetry model and/or simulated augmented fetal oximetry model may be determined, set, and/or selected for input into a machine learning program and/or architecture such as TensorFlow. In some embodiments, execution of step 710 may resemble execution of step 410 and/or 515. Then, in step 715, a simulated fetal oximetry model and/or simulated augmented fetal oximetry model may be trained using the simulated light transmission data sets and corresponding oximetry values. Step 715 may be executed via, for example, inputting the simulated light transmission data and corresponding oximetry values into the machine learning architecture once it is set up with the machine learning inputs of step 710. At times, execution of step 715 may resemble execution of step 520. Additionally, or alternatively, in some embodiments, execution of step 715 may include training the augmented in vivo fetal oximetry model using the additional information received in step 705 and/or information (e.g., evaluations, etc.) correlated thereto so that, for example, the augmented in vivo fetal oximetry model may be used to provide one or more evaluations of fetal health based upon fetal oximetry values, the additional information, and/or correlations between the fetal oximetry values and evaluations thereof and/or correlations between the fetal oximetry values and values for physiological indicators and/or evaluations of the values for physiological indicators.

The simulated fetal oximetry model and/or simulated augmented fetal oximetry model may be trained and/or configured to receive a plurality of sets of simulated light transmission data and determine an oximetry value for a fetus that may be associated with each set of simulated light transmission data. This determined oximetry value may then be compared with the oximetry value associated with respective sets of simulated light transmission data received in step 705 to determine any differences therebetween. Results of this comparison may be used to iteratively update/train the simulated fetal oximetry model and/or simulated augmented fetal oximetry model during execution of step 715. Training of the simulated fetal oximetry model and/or simulated augmented fetal oximetry model may be complete (step 720) when, for example, a number or proportion (e.g., 60-99%) of the oximetry values and/or fetal health indicators calculated by the simulated fetal oximetry model and/or simulated augmented fetal oximetry model using one or more simulated light transmission data sets and/or one or more simulated light transmission data sets and corresponding values for physiological indicators received in step 705 are sufficiently close to (e.g., within a standard of deviation, within 0.5 standards of deviation, within 0.1 standards of deviation, and/or within 60-99% of the associated oximetry value) the oximetry values and/or fetal health indicators associated each of the respective simulated light transmission data sets. When the training of the simulated fetal oximetry model and/or simulated augmented fetal oximetry model is not complete (step 720), step 715 may be iteratively repeated as needed. In some embodiments, execution of step 720 may resemble execution of step 525.

In some embodiments, the simulated fetal oximetry model and/or simulated augmented fetal oximetry model includes a plurality of layers, factors, calibrations, and/or functions (referred to herein collectively as “layers”) that are used to calculate oximetry values using the simulated light transmission data. Layers may include, for example, functions that account for and/or factor in, for example, fetal depth, source/detector separation distance, a thickness of maternal tissue, a type of maternal tissue, maternal and/or fetal skin color and/or melanin content, a thickness of fetal tissue, a type of fetal tissue, a type of light used, an intensity of light used, a fetal age, and/or calibration factor(s) associated with equipment that may be used in clinical applications to obtain in vivo measurements of light transmission data, environmental conditions that may be present during clinical applications when in vivo measurements of light transmission data is collected. When the training of the simulated fetal oximetry model and/or simulated augmented fetal oximetry model is complete (step 720), it may be stored in a database like database 15 and/or 170 (step 725).

In step 730, instructions to adapt the simulated fetal oximetry model and/or simulated augmented fetal oximetry model for transfer to an in vivo fetal oximetry model and/or augmented in vivo fetal oximetry model may then be received. In some cases, the instructions to adapt the simulated fetal oximetry model and/or simulated augmented fetal oximetry model for transfer to an in vivo fetal oximetry model and/or augmented in vivo fetal oximetry model may include instructions to fix, lock, or otherwise prevent changes to, one or more layers, or functions, of the simulated fetal oximetry model and/or simulated augmented fetal oximetry model that may be generally applicable to the in vivo fetal oximetry model and/or augmented in vivo fetal oximetry model so that these fixed/locked layers/functions do not change during the training process. Exemplary layers and/or functions of the simulated fetal oximetry model and/or simulated augmented fetal oximetry model that may be fixed and/or locked include, but are not limited to, how one or more of a source/detector distance, a wavelength of light, a fetal depth, maternal skin color, fetal skin color, maternal tissue composition, fetal tissue composition, value for a physiological indicator, an evaluation of a value for a physiological indicator, and/or a calibration factor impact (e.g., weights in the model), an oximetry calculation and/or determination of a fetal health indicator. In some embodiments, execution of step 730 may resemble execution of step 424 and/or 610.

Then, the simulated fetal oximetry model and/or simulated augmented fetal oximetry model may be adapted for transfer to an in vivo fetal oximetry model and/or augmented in vivo fetal oximetry model responsively to the instructions (step 735). In some cases, the adapting of step 735 may include determining, setting, and/or selecting one or more machine learning inputs for a machine learning architecture for the generation of an in vivo fetal oximetry model and/or augmented in vivo fetal oximetry model. Additionally, or alternatively, the adapting of step 735 may include fixing one or more layers, or functions, of the simulated fetal oximetry model and/or simulated augmented fetal oximetry model so that it remains fixed during the in vivo fetal oximetry model and/or augmented in vivo fetal oximetry model training and/or tuning process (step 745, which is discussed below).

In step 740, a plurality (e.g., 1,000-10 million) of sets of in vivo light transmission data and corresponding fetal oximetry values and optional values for physiological indicators for each set of in vivo light transmission may be received. The plurality of sets of in vivo light transmission data may be received from, for example, a fetal oximetry probe like fetal oximetry probe 115 and the corresponding fetal oximetry values and/or fetal health indicators may be calculated using, for example, Equations 1 and 2 as discussed herein. Then, in step 745, an in vivo fetal oximetry model and/or augmented in vivo fetal oximetry model may be generated and/or trained using the in vivo data and the adapted simulated fetal oximetry model and/or simulated augmented fetal oximetry model of step 735. Step 745 may be executed via, for example, inputting a plurality of sets of in vivo data and, where appropriate, corresponding sets of values for physiological indicators into the machine learning architecture once it is set up with the adapted simulated fetal oximetry model and/or simulated augmented fetal oximetry model of step 735. The in vivo fetal oximetry model and/or augmented in vivo fetal oximetry model may be configured to receive a plurality of sets of in vivo light transmission data and, where appropriate, corresponding sets of values for physiological indicators and determine an oximetry value and/or fetal health indicator of a fetus for each set of in vivo light transmission data included in the training set of in vivo data. This determined oximetry value and/or fetal health indicator may then be compared with the oximetry value and/or fetal health indicator associated with a respective set of in vivo light transmission data that may be received in step 740 to determine any differences therebetween. These differences may be used to, for example, iteratively update/train the in vivo fetal oximetry model and/or augmented in vivo fetal oximetry model during execution of step 745. Training of the in vivo fetal oximetry model and/or augmented in vivo fetal oximetry model may be complete (step 750) when, for example, a number or proportion (e.g., 60-99%) of the oximetry values calculated by the in vivo fetal oximetry model and/or augmented in vivo fetal oximetry model using one or more in vivo light transmission data sets received in step 740 are sufficiently close to (e.g., within a standard of deviation, within 0.5 standards of deviation, within 0.1 standards of deviation, and/or within 60-99% of the associated oximetry value) to the oximetry values associated with each of the respective in vivo transmitted light data sets.

In some embodiments, the in vivo fetal oximetry model and/or augmented in vivo fetal oximetry model includes a plurality of layers, factors, calibrations, and/or functions (referred to herein collectively as “layers”) that are used to calculate oximetry values using the in vivo light transmission data. Exemplary layers include functions that factor in and/or account for associated with, for example, fetal depth, source/detector separation distance, a thickness of maternal tissue, a type of maternal tissue, maternal and/or fetal skin color and/or melanin content, a thickness of fetal tissue, a type of fetal tissue, a type of light used, an intensity of light used, a fetal age, and/or calibration factor(s) associated with equipment used to obtain the in vivo light transmission data, environmental conditions when the in vivo light transmission data is collected.

When the training of the in vivo fetal oximetry model and/or augmented in vivo fetal oximetry model is not complete, (step 745) may be repeated and/or may continue to be iteratively executed. When the training of the in vivo fetal oximetry model and/or augmented in vivo fetal oximetry model is complete (step 745), the in vivo fetal oximetry model and/or augmented in vivo fetal oximetry model may then be finalized and stored (step 755) and process 700 may end or proceed to step 805 of process 800 discussed below.

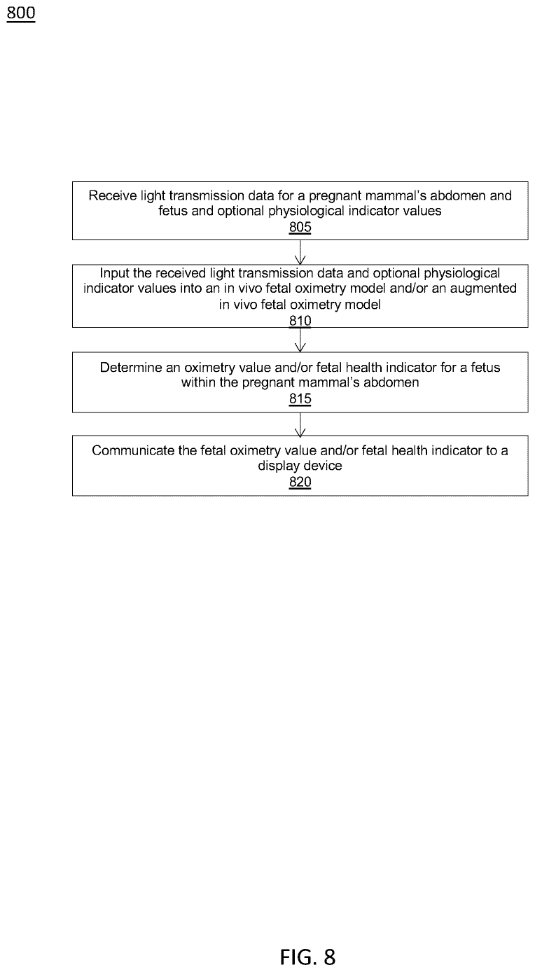

FIG. 8 is a flowchart illustrating an exemplary process 800 for the determination of a fetal oximetry value and/or a fetal health indicator for a fetus using an in vivo fetal oximetry model and/or augmented in vivo fetal oximetry model that may be generated via, for example, execution of process 600 and/or 700. Process 800 may be performed by, for example, any of the systems or system components disclosed herein.

Initially, in step 805, light transmission data for a pregnant mammal's abdomen and fetus may be received from, for example, a photodetector like detector 160 and/or a probe like fetal oximetry probe 115. The light may be transmitted from a light source, through the pregnant mammal's abdomen and fetus and/or backscattered from the abdominal/fetal tissue and detected by the photodetector. Optionally, values for one or more physiological indicators for the pregnant mammal and/or fetus may also be received in step 805. The light transmission data and optional values for one or more physiological indicators received in step 805 may then be put into and/or processed by the in vivo fetal oximetry model and/or augmented in vivo fetal oximetry model (which may be the finalized in vivo fetal oximetry model and/or augmented in vivo fetal oximetry model of step 650 and/or 755) in step 810. In some embodiments, the light transmission data and/or values for one or more physiological indicators received in step 805 may be pre-processed prior to execution of step 810. The pre-processing may include, for example, filtering with, for example, a Kalman or bandpass filter, application of a noise reduction model, removal of a portion of the light transmission data that is incident only the pregnant mammal (i.e., not incident on the fetus), and/or isolation of a portion of the light incident on the fetus from the received light transmission data. On some occasions, removal of a portion of the light transmission data that is incident only the pregnant mammal (i.e., not incident on the fetus), and/or isolation of a portion of the light incident on the fetus from the received light transmission data may be accomplished by, for example, receiving a maternal heartrate signal, using the maternal heart rate signal to identify the portion of the light transmission data contributed by the pregnant mammal and then subtracting the portion of the light transmission data contributed by the pregnant mammal from the light transmission data. Additionally, or alternatively, isolation of the fetal portion of the light transmission data may be accomplished by, for example, receiving a fetal heartrate signal, using the fetal heart rate signal to identify the portion of the light transmission data contributed by the fetus and then subtracting the remainder of light transmission data and/or amplifying the portion of the light transmission data contributed by the fetus. Additionally, or alternatively, isolation of the fetal portion of the light transmission data may include determining a fetal position and/or fetal depth and then

In step 815, an oximetry value and/or fetal health indicator for the fetus within the pregnant mammal's abdomen may be determined and/or output by the in vivo fetal oximetry model and/or augmented in vivo fetal oximetry model. The oximetry value and/or fetal health indicator may be, for example, a fetal hemoglobin oxygen saturation level, a fetal tissue oxygen saturation level, an indication of fetal hypoxia, an indication of fetal hypoxemia, and/or an alert condition indicating that a fetus may be in distress. The oximetry value and/or fetal health indicator may then be communicated to a display device like display device 14 and/or 155 for display to a user such as a clinician and/or the pregnant mammal (step 820).

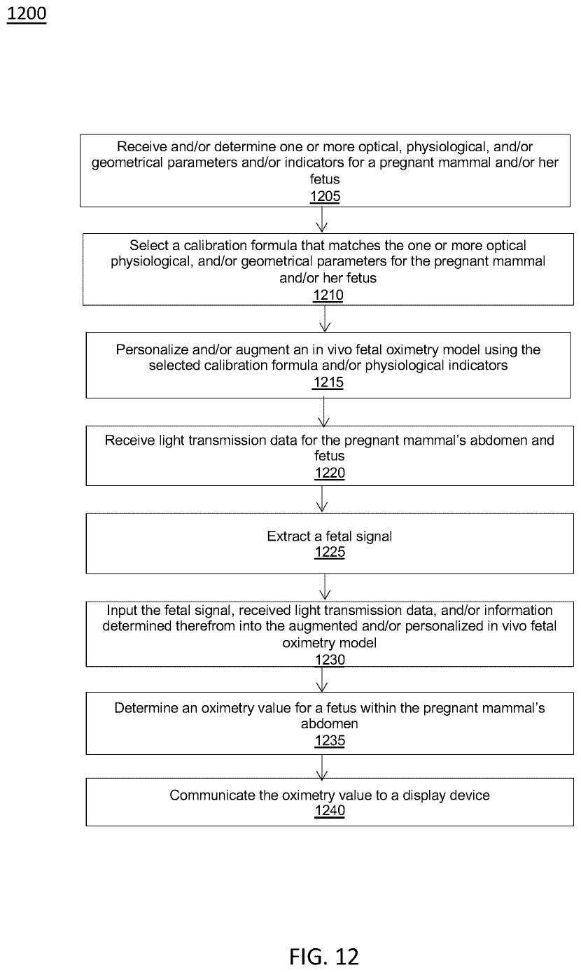

FIG. 12 provides a flowchart of an exemplary process 1200 for using an in vivo fetal oximetry model and/or augmented in vivo fetal oximetry model to determine a fetal oxygenation value and/or fetal health indicator. Process 1200 may be executed by, for example, any of the systems or system components disclosed herein.

Initially, one or more optical, physiological, and/or geometrical properties, parameters, and/or indicators of a pregnant mammal and/or her fetus may be received and/or determined (step 1205). Exemplary optical features include, but are not limited to, light scattering and/or light absorption coefficients for the maternal and/or fetal tissue that may be known and/or determined via, for example, execution of a frequency domain (e.g., FFT) analysis of an optical signal corresponding to light that has traveled through the maternal abdomen and/or analysis of time of flight for photons detected upon emission from a pregnant mammal's abdomen. Exemplary physiological parameters include, but are not limited to, maternal oximetry information, maternal and/or fetal skin color, and maternal body mass index. Exemplary physiological indicators include, but are not limited to, fetal heart rate, uterine contraction information, and fetal ECG information. Exemplary geometrical features include, but are not limited to, fetal depth, a thickness of one or more layers of maternal tissue the light passes through, a part of the fetus (e.g., head, back, face, etc.) light is incident upon, and a fetal position.

On some occasions, fetal depth may be deduced using, for example, relative distances between a light source and one or more detectors that detect light transmission data that includes light incident upon the fetus. For example, in an array of four detectors placed in a linear configuration at a distance of 1 cm, 2 cm, 3 cm, and 4 cm, respectively, if the second detector detects light transmission data that includes light incident upon the fetus it may be deduced that the fetus is relatively shallow (i.e., fetal depth is relatively small) and using the geometry of the source/detector distance between the light source and the second detector, a fetal depth may be deduced. Likewise, if only the fourth detector detects light transmission data that includes light incident upon the fetus it may be deduced that the fetus is relatively deep (i.e., fetal depth is relatively large) and using the geometry of the source/detector distance between the light source and the fourth detector, a fetal depth may be deduced.

On some occasions, light absorption by the pregnant mammal and/or fetus may be responsive to skin pigmentation and/or a level of melanin in the skin of the pregnant mammal and/or fetus. At times, skin color may be received in step 1205 to assist with the determination of light absorption characteristics (e.g., absorption and/or scattering) of the pregnant mammal and/or fetus and/or the selection or application of a calibration factor or function to apply to light transmission data and/or received optical and/or detected electronic signals.

Additionally, or alternatively, light scattering properties of the pregnant mammal may be a function of tissue layer composition (e.g., skin, adipose, muscle) and relative thicknesses of the tissue layers for her abdomen. This information may be provided by, for example, an imaging technique such as ultrasound and/or MRI scan.

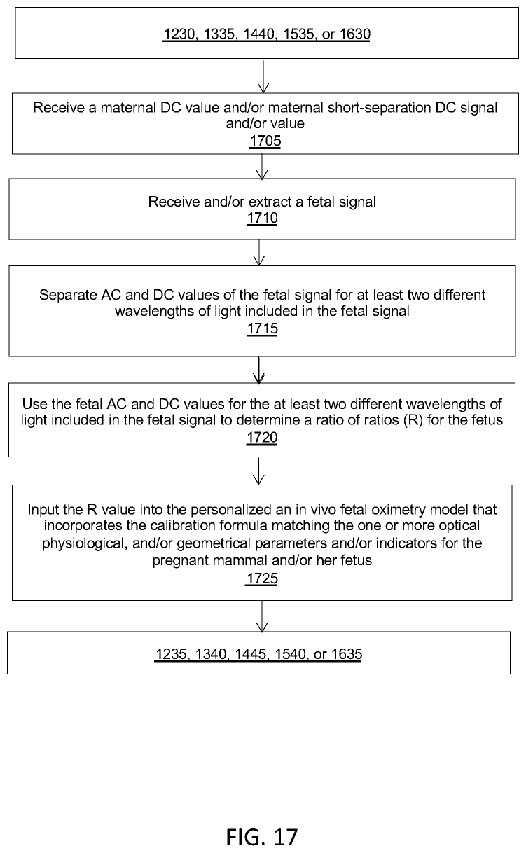

In step 1210, a calibration formula, such as the calibration formulas defining the best fit lines shown in graphs 201 and/or 202, that match the one or more optical, physiological, and/or geometrical parameters of a pregnant mammal and/or her fetus received and/or determined in step 1205 may be determined. At times, execution of step 1210 includes querying a memory storing various calibration formulas and/or a database of and/or calibration formulas such as database 15 and/or 170 or a portion thereof for a calibration formula that matches some or all of the parameters and/or indicators of step 1205. The selected calibration formula may be used to augment and/or personalize an in vivo fetal oximetry model (which may generate an augmented in vivo fetal oximetry model) to the pregnant mammal and/or fetus (step 1215). At times, execution of step 1215 may include adjusting one or more inputs and/or processes of the in vivo fetal oximetry model and/or augmented in vivo fetal oximetry model. Further details on how step 1215 may be performed are provided below with regard to process 1700 of FIG. 17.

In step 1220, light transmission data (e.g., a detected electronic signal a voltage signal, a set of detected electronic signals, and/or a set of voltage signals, that corresponds to an optical signal of two or more wavelengths) for light that has emanated from the pregnant mammal's abdomen and fetus may be received from, for example, a fetal oximetry probe like fetal oximetry probe 115 and/or 117. The light transmission data may be processed to isolate a fetal signal (step 1225) that corresponds to light that was incident upon the fetus. Further details regarding how step 1225 may be performed are provided below with regard to processes 1700 and/or 1900 of FIGS. 17 and/or 19, respectively.

The fetal signal received light transmission data, and/or information determined therefrom may then be input into the personalized in vivo fetal oximetry model and/or augmented in vivo fetal oximetry model (step 1230), an oximetry value for the fetus may be determined (step 1235), and the oximetry value for the fetus may be communicated to a display device for observation by, for example, a clinician and/or the pregnant mammal (step 1240). In some instances, personalizing the in vivo fetal oximetry model and/or augmented in vivo fetal oximetry model may include, for example, adding, subtracting, and/or modifying one or more features, portions, and/or formulas of the in vivo fetal oximetry model and/or augmented in vivo fetal oximetry model to incorporate, or factor in, data relating to the pregnant mammal and/or fetus that may be received in, for example, step 1205. This personalization of the in vivo fetal oximetry model and/or augmented in vivo fetal oximetry model enables more accurate calculation of a fetal oximetry value in step 1235 because, for example, the calculation incorporates features specific to the particular pregnant mammal and fetus being studied. Further details regarding how steps 1230 and 1235 may be performed are provided below with regard to process 1700 of FIG. 17.

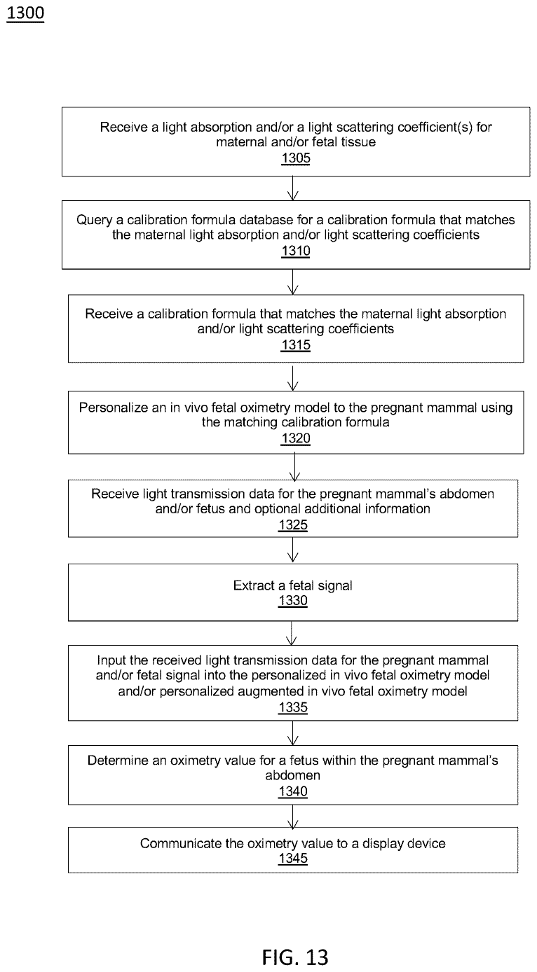

FIG. 13 provides a flowchart of an exemplary process 1300 for selecting a calibration formula for use with an in vivo fetal oximetry model and/or augmented in vivo fetal oximetry model and determine a fetal oxygenation value using the calibration formula and an in vivo fetal oximetry model and/or augmented in vivo fetal oximetry model. Process 1300 may be executed by, for example, any of the systems or system components disclosed herein.

Initially, light scattering and/or light absorption coefficients for the maternal and/or fetal tissue that may be received (step 1305). In some embodiments, the light scattering and/or light absorption coefficients for the maternal tissue may be determined via frequency domain analysis (e.g., FFT) and/or analysis of time of flight for of an optical signal corresponding to light that only passes through maternal tissue as may be the case with, for example, a short-separation measurement. At times this optical signal and/or a digital signal corresponding to it may be received from first light/source detector system 107.

In step 1310, a database of various calibration formulas (e.g., the calibration formulas of graphs 201 and 202) such as database 15 and/or 170 or a portion thereof, may be queried for a calibration formula that matches light scattering and/or light absorption coefficients for the maternal and/or fetal tissue of step 1305. Often times, the query of step 1310 may specify that the returned calibration formula must match both the light scattering coefficient and the light absorption coefficient for the maternal tissue. In step 1315, a calibration formula that matches light scattering and/or light absorption coefficients for the maternal and/or fetal tissue may be received and used to personalize an in vivo fetal oximetry model and/or augmented in vivo fetal oximetry model to the pregnant mammal and/or fetus (step 1320). At times, execution of step 1320 may include adjusting one or more inputs, subroutines, and/or processes of the in vivo fetal oximetry model and/or augmented in vivo fetal oximetry model to personalize it to, for example, the pregnant mammal's and/or fetus' light scattering and/or light absorption coefficients and/or calibration curves that may be specific to, for example, the skin tone (e.g., melanin content) of the pregnant mammal and/or fetus. Further details on how step 1320 may be performed are provided below with regard to process 1700 of FIG. 17.

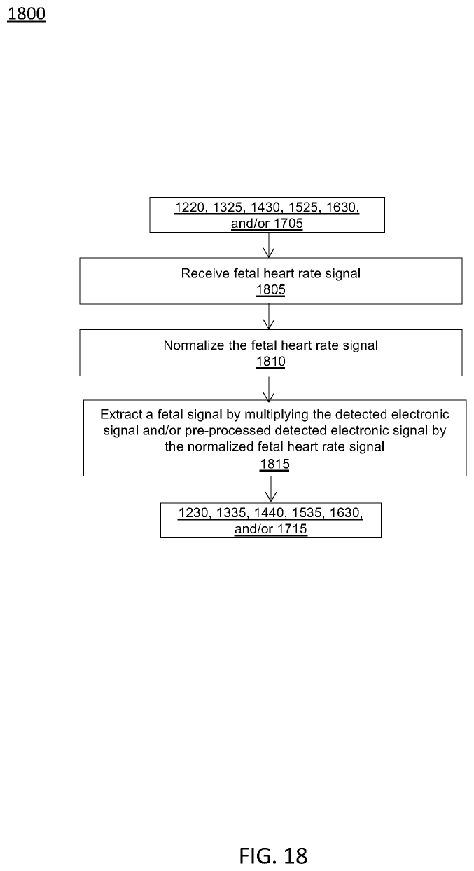

In step 1325, light transmission data (e.g., a detected electronic signal that corresponds to an optical signal of two or more wavelengths) for light that has emanated from the pregnant mammal's abdomen, and, in some cases, the fetus may be received from, for example, a fetal oximetry probe like fetal oximetry probe 115 and/or 117 and/or second source/detector system 167. In some embodiments, additional information such as physiological indicators and/or measurements may also be received in step 1325. The light transmission data may be processed to isolate a fetal signal (step 1330) that corresponds to light that was incident upon the fetus. At times, step 1330 may be performed using information received in step 1325. Further details regarding how step 1330 may be performed are provided below with regard to processes 1800 and/or 1900 of FIGS. 18 and/or 19, respectively.

The fetal signal, received light transmission data, optional additional information, and/or information determined therefrom may then be input into the personalized in vivo fetal oximetry model and/or augmented in vivo fetal oximetry model (step 1335), an oximetry value for the fetus and/or fetal health indicator may be determined (step 1340), and the oximetry value for the fetus may be communicated to a display device for observation by, for example, a clinician and/or the pregnant mammal (step 1345). Further details regarding how steps 1340 and 1345 may be performed are provided below with regard to process 1700 of FIG. 17.

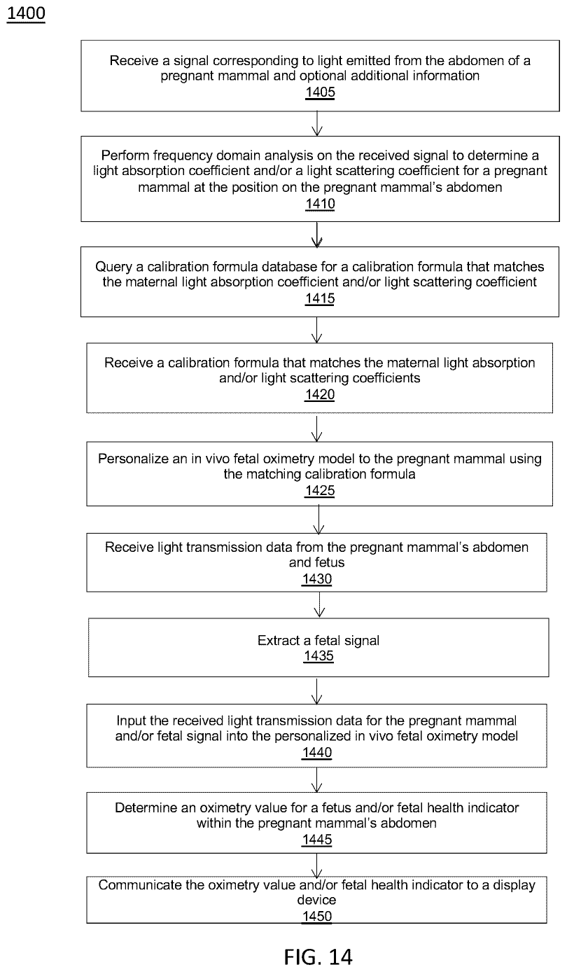

FIG. 14 provides a flowchart of an exemplary process 1400 for determining optical properties of maternal tissue, selecting a calibration formula for use with an in vivo fetal oximetry model and/or augmented in vivo fetal oximetry model responsively to the maternal optical properties, and determining a fetal oxygenation value and/or fetal health indicator using the calibration formula and an in vivo fetal oximetry model and/or augmented in vivo fetal oximetry model. Process 1400 may be executed by, for example, any of the systems or system components disclosed herein.

Initially, a signal corresponding to light emitted from the abdomen of a pregnant mammal (also referred to herein as light transmission data) may be received (step 1405). In some cases, the signal received in step 1405 may not include light that was incident on the fetus and may be, for example, a short separation signal that only penetrates maternal tissue. In some embodiments, additional information such as physiological indicators and/or measurements may also be received in step 1405.

In step 1410, frequency domain (e.g., FFT) analysis and/or analysis of time of flight for photons detected upon emission from a pregnant mammal's abdomen may be performed on the received signal to determine light scattering and/or light absorption coefficients for the maternal tissue. In some cases, execution of step 1410 may also determine a skin tone, or level of melanin content, in the skin of the pregnant mammal and/or fetus. In some embodiments, a position of a probe providing the signal received in step 1405 may also be received in step 1405 and this position may be used to, for example, determine the optical properties of the pregnant mammal at a position near and/or at a known distance from where the light transmission data corresponding to light that was incident on the fetus.

In step 1415, a database of various calibration formulas (e.g., such the calibration formulas of graph 201 and 202), such as database 15 and/or 170 or a portion thereof, may be queried for a calibration formula that matches light scattering and/or light absorption coefficients and/or melanin level for the maternal tissue and/or fetal tissue. Often times, the query of step 1415 may specify that the returned calibration formula must match both the light scattering coefficient and the light absorption coefficient for the maternal tissue. In step 1420, a calibration formula that matches light scattering and/or light absorption coefficients for the maternal and/or fetal tissue may be received and used to personalize an in vivo fetal oximetry model and/or augmented in vivo fetal oximetry model to the pregnant mammal and/or fetus (step 1425). At times, execution of step 1425 may include adjusting one or more inputs, subroutines, and/or processes of the in vivo fetal oximetry model and/or augmented in vivo fetal oximetry model to personalize it to the pregnant mammal's and/or fetus' light scattering and/or light absorption coefficients. Further details on how step 1425 may be performed are provided below with regard to process 1700 of FIG. 17.

In step 1430, light transmission data (e.g., a detected electronic signal that corresponds to an optical signal of two or more wavelengths) for light that has emanated from the pregnant mammal's abdomen and fetus may be received from, for example, a fetal oximetry probe like fetal oximetry probe 115 and/or 117 and/or second source/detector system 167. The light transmission data may be processed to isolate a fetal signal (step 1435) that corresponds to light that was incident upon the fetus. Further details regarding how step 1435 may be performed are provided below with regard to processes 1800 and/or 1900 of FIGS. 18 and/or 19, respectively.

The fetal signal, received light transmission data, additional information (received e.g., in step 1405) and/or information determined therefrom may then be input into the personalized in vivo fetal oximetry model and/or augmented in vivo fetal oximetry model (step 1440), an oximetry value and/or fetal health indicator for the fetus may be determined (step 1445), and the oximetry value and/or fetal health indicator for the fetus may be communicated to a display device for observation by, for example, a clinician and/or the pregnant mammal (step 1450). Further details regarding how steps 1445 and 1450 may be performed are provided below with regard to process 1700 of FIG. 17.

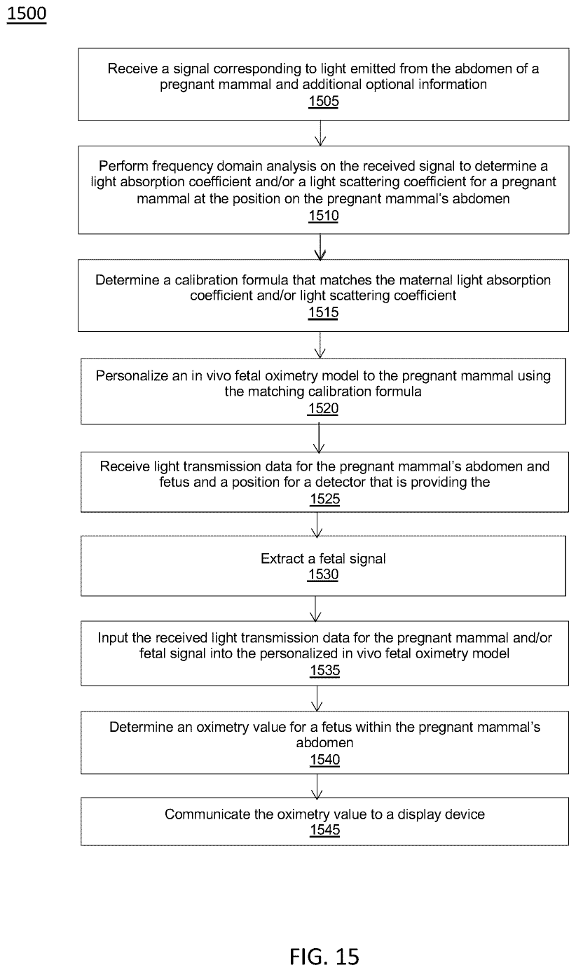

FIG. 15 provides a flowchart of another exemplary process 1500 for determining optical properties of maternal and/or fetal tissue, selecting a calibration formula for use with an in vivo fetal oximetry model and/or augmented in vivo fetal oximetry model responsively to the maternal and/or fetal optical properties, and determining a fetal oxygenation value and/or fetal health indicator using the calibration formula and an in vivo fetal oximetry model and/or augmented in vivo fetal oximetry model. Process 1500 may be executed by, for example, any of the systems or system components disclosed herein.

Initially, a signal corresponding to light emitted from the abdomen of a pregnant mammal (also referred to herein as light transmission data) may be received (step 1505). In some cases, the signal received in step 1505 may not include light that was incident on the fetus and may be, for example, a short separation signal that only penetrates maternal tissue. In some embodiments, additional information such as physiological indicators and/or measurements may also be received in step 1505.

In step 1510, frequency domain (e.g., FFT) analysis and/or analysis of time of flight for photons detected upon emission from a pregnant mammal's abdomen may be performed on the received signal to determine light scattering and/or light absorption coefficients for the maternal and/or fetal tissue and/or melanin levels (i.e., skin tone) for the maternal and/or fetal skin. In some embodiments, a position of a probe providing the signal received in step 1505 may also be received in step 1505. In some embodiments, a position of a probe providing the signal received in step 1505 may also be received in step 1505 and this position may be used to, for example, determine the optical properties of the pregnant mammal at a position near and/or at a known distance from where the light transmission data corresponding to light that was incident on the fetus.

In step 1515, a calibration formula (e.g., such the calibration formulas of graph 201 and 202) may be calculated or otherwise determined using, for example, the light scattering and/or light absorption coefficients, or other optical properties for the maternal tissue. The calibration formula determined in step 1515 may then be used to personalize an in vivo fetal oximetry model and/or augmented in vivo fetal oximetry model to the pregnant mammal (step 1520). At times, execution of step 1520 may include adjusting one or more inputs, subroutines, and/or processes of the in vivo fetal oximetry model and/or augmented in vivo fetal oximetry model to personalize it to the pregnant mammal's skin tone, light scattering coefficients, and/or light absorption coefficients. Further details on how step 1520 may be performed are provided below with regard to process 1700 of FIG. 17.

In step 1525, light transmission data (e.g., a detected electronic signal that corresponds to an optical signal of two or more wavelengths) for light that has emanated from the pregnant mammal's abdomen and fetus may be received from, for example, a fetal oximetry probe like fetal oximetry probe 115 and/or 117 and/or second source/detector system 167. The light transmission data may be processed to isolate a fetal signal (step 1530) that corresponds to light that was incident upon the fetus. Further details regarding how step 1530 may be performed are provided below with regard to processes 1800 and/or 1900 of FIGS. 18 and/or 19, respectively.

The fetal signal, received light transmission data, additional information received in step 1505, and/or information determined therefrom may then be input into the personalized in vivo fetal oximetry model and/or augmented in vivo fetal oximetry model (step 1535), an oximetry value for the fetus may be determined (step 1540), and the oximetry value for the fetus may be communicated to a display device for observation by, for example, a clinician and/or the pregnant mammal (step 1545). Further details regarding how steps 1540 and 1545 may be performed are provided below with regard to process 1700 of FIG. 17.

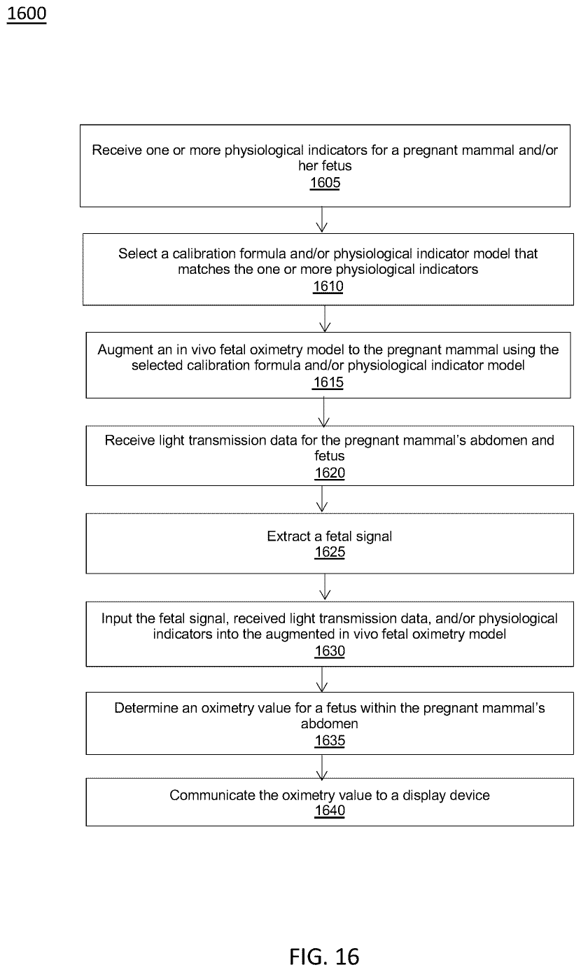

FIG. 16 provides a flowchart of an exemplary process 1600 for using an augmented in vivo fetal oximetry model to determine a fetal oxygenation value and/or fetal health indicator. Process 1600 may be executed by, for example, any of the systems or system components disclosed herein.

Initially, one or more physiological indicators for a pregnant mammal and/or her fetus may be received and/or determined (step 1605). Exemplary physiological indicators include, but are not limited to, maternal and/or fetal hemoglobin oxygen saturation level, tissue oxygen saturation level, skin tone, heartrate, and ECG information. Additional, or alternative, physiological indicators include maternal blood pressure, maternal photoplethysmogram (PPG) signals or values, maternal pulse, maternal respiratory rate, uterine contraction information, duration of labor and delivery of a fetus, gestational age of the fetus, chronological age of the pregnant mammal, length of time the pregnant mammal is in labor, and a stage of labor and delivery. The physiological indicators may be received on a one-time, periodic, and/or as-needed basis.

In step 1610, a calibration formula and/or physiological indicator model that matches the one or more physiological indicators received in step 1605 may be selected and/or determined in step 1610. At times, execution of step 1610 includes querying a memory or database of calibration formulas and/or physiological indicator models such as database 15 and/or 270 for one or more calibration formulas and/or physiological indicator models that matche some, or all, of the physiological indicators received in step 1605. The selected calibration formula and/or physiological indicator model may then be input into an in vivo fetal oximetry model to, for example, augment and/or personalize an in vivo fetal oximetry model to the pregnant mammal and/or fetus (step 1615). At times, execution of step 1615 may include adjusting one or more inputs, sub-routines, and/or processes of the in vivo fetal oximetry model and/or generation of an augmented in vivo fetal oximetry model.

In step 1620, light transmission data (e.g., a detected electronic signal a voltage signal, a set of detected electronic signals, and/or a set of voltage signals, that corresponds to an optical signal of two or more wavelengths) for light that has traveled through and/or emanated from the pregnant mammal's abdomen and fetus may be received from, for example, a fetal oximetry probe like fetal oximetry probe 215 and/or 217. The light transmission data may be processed to isolate, or extract, a fetal signal (step 1625) that corresponds to light that was incident upon the fetus.

The fetal signal received light transmission data, and/or the one or more physiological indicators may then be input into the augmented in vivo fetal oximetry model (step 1630) and an oximetry value and/or wellness indicator for the fetus may be determined as an output from the augmented in vivo fetal oximetry model (step 1635). Then, the oximetry value for the fetus may be communicated to a display device for observation by, for example, a clinician and/or the pregnant mammal (step 1640). In some instances, augmenting and/or personalizing the fetal oximetry model may include, for example, adding, subtracting, and/or modifying one or more features, portions, and/or formulas of a fetal oximetry model to incorporate, or factor in, data relating to the physiological indicators of the pregnant mammal and/or fetus that may be received in, for example, step 1605. This augmentation and/or personalization of the vivo fetal oximetry model enables more accurate calculation of a fetal oximetry value in step 1635 because, for example, the calculation incorporates features specific to the particular pregnant mammal and fetus being studied. Further details regarding how steps 1630 and 1635 may be performed are provided below with regard to process 1700 of FIG. 17.

FIG. 17 provides a flowchart of an exemplary process 1700 for determining a fetal oximetry value and/or fetal health indicator using a calibration formula and an in vivo fetal oximetry model and/or augmented in vivo fetal oximetry model. Process 1700 may be executed by, for example, any of the systems or system components disclosed herein following, for example, execution of steps 1230, 1335, 1440, or 1535, of process 1200, 1300, 1400, or 1500 respectively.

Initially, in step 1705, a maternal DC value or signal and/or a maternal short-separation DC signal may be received. Optionally, in step 1705, the maternal DC value may be received from, for example, a pulse oximeter like pulse oximetry probe 130 and/or NIRS adult hemoglobin probe 125. A fetal signal may then be received and/or generated (step 1710) via one or more of the processes described herein. In many cases, the fetal signal includes an AC and a DC value for multiple wavelengths of light. The fetal AC and DC values may then be extracted from the fetal signal (step 1715). On some occasions, execution of step 1715 may include subtracting all AC signals and the maternal DC signal from a DC portion of light transmission data corresponding to light emanating from the pregnant mammal's abdomen such as the light transmission data received in step 1220, 1325, 1430, and 1525; with the remainder of the DC portion of the light transmission data being a fetal DC signal for multiple wavelengths of light. Additionally, or alternatively, separating the AC values of the fetal signal may include subtracting all DC signals and the maternal AC signal from the light transmission data; with the remainder being the AC signals, or values, being fetal AC signals or values for multiple wavelengths. Then, in step 1720, a ratio of ratios (R) may be calculated for the fetus according to, for example, Equation 3, below:

$$R = \frac{AC / DC ( λ1 )}{AC / DC ( λ2 )} \qquad Equation 3$$

The R value of step 1720 may then be input into a personalized in vivo fetal oximetry model and/or augmented in vivo fetal oximetry model as part of the execution of steps 1235, 1340, 1445, or 1540 of process 1200, 1300, 1400, or 1500, respectively, to determine an oximetry value for the fetus and/or an indicator of fetal health (step 1725). Then, steps 1235, 1340, 1445, and/or 1540 may be executed.

FIG. 18 provides a flowchart of an exemplary process for generating a fetal signal. Process 1800 may be executed by, for example, any of the systems or system components disclosed herein.

Following execution of step 1220, 1325, 1430, 1525, and/or 1705, a fetal heart rate signal may be received from, for example, a Doppler/ultrasound probe like Doppler/ultrasound probe 135 and/or an ECG like ECG 175 (step 1805). Optionally, the fetal heart rate signal may be normalized (step 1810) and/or synchronized, in time, with, for example, the light transmission data and/or fetal signal. In some embodiments, the normalization of step 1810 may include adjusting values of one or more measurements and/or components of the detected electronic signal (e.g., intensity magnitudes for different wavelengths of light) to be on a similar, or common, scale so that the different values may be more easily evaluated/analyzed. The fetal heart rate signal of step 1805 or the normalized fetal heart rate signal of step 1810 may then be multiplied by the light transmission data received in, for example, step 1220, 1325, 1430, and 1525 of process 1200, 1300, 1400, or 1500, respectively, to generate the fetal signal (step 1815).

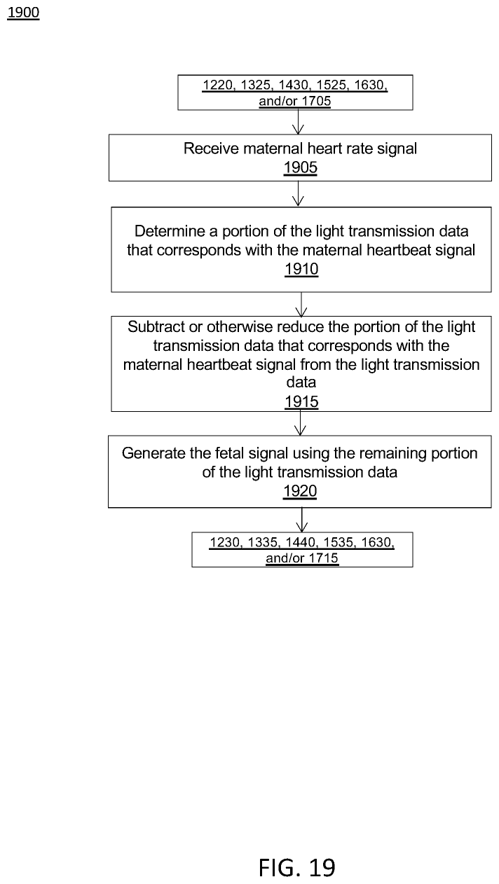

FIG. 19 provides a flowchart of another exemplary process for generating a fetal signal. Process 1900 may be executed by, for example, any of the systems or system components disclosed herein.

In step 1905, a maternal heart rate signal may be received and a portion of the fetal signal of step 1815 and/or light transmission data received in, for example, step 1220, 1325, 1430, and 1525 of process 1200, 1300, 1400, or 1500, respectively may be analyzed to determine a portion of there of that corresponds to the heartbeat signal of the pregnant mammal (step 1910). At times, this analysis may include synchronizing the maternal heart rate signal and the fetal signal of step 1815 and/or light transmission data in time and then comparing the fetal signal and/or light transmission data with the heartbeat signal of the pregnant mammal. Then, the portion of the fetal signal of step 1815 and/or light transmission data that corresponds to the heart beat signal of the pregnant mammal may be subtracted from, regressed out of the portion of the multiplied signal via, for example, a linear regression expression, or otherwise reduced, or removed, from the multiplied signal (step 1915) and the fetal signal of step 1815 may be generated using the remaining portion of the fetal signal of step 1815 and/or light transmission data (step 1920). Following execution of step 1920, step 1220, 1325, 1430, and 1525 of process 1200, 1300, 1400, or 1500, respectively, may be executed.

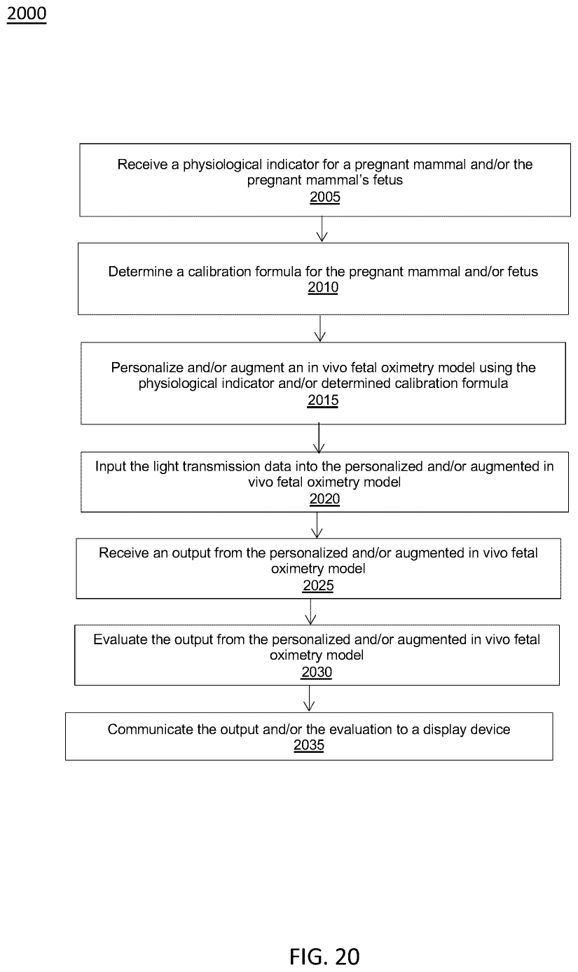

FIG. 20 provides a flowchart of an exemplary process 2000 for determining an indication of an oximetry value for a fetus. Process 2000 may be executed by, for example, any of the systems or system components disclosed herein.

In step 2005, a physiological indicator for a pregnant mammal and/or her fetus may be received. Exemplary physiological indicators include, but are not limited to, a hemoglobin oxygen saturation level, a tissue oxygen saturation level, a skin tone, a heartrate, a blood pressure, photoplethysmogram (PPG) signals or values, pulse, respiratory rate, uterine contraction information, duration of labor and delivery of a fetus, gestational age of the fetus, chronological age of the pregnant mammal, a hemoglobin oxygen saturation level, and a tissue oxygenation level for the pregnant mammal and/or fetus.

In step 2010, light transmission data may be received. The light transmission data may correspond to an optical signal that is detected by a photodetector and converted into the light transmission data. The optical signal may be a composite of light that passes through the pregnant mammal's abdomen and a fetus disposed within the pregnant mammal's abdomen.

Optionally, in step 2010, a calibration formula for the pregnant mammal and/or fetus may be determined using, for example, the physiological indicator received in step 2005. In some embodiments, step 2010 may be executed by querying a database of calibration equations for a calibration equation for the pregnant mammal and/or fetus, the query requesting a calibration equation that is responsive to the physiological indicator of the pregnant mammal and/or fetus. A calibration equation may then be responsively received from the database. Additionally, or alternatively, a calibration formula that incorporates the physiological indicator may be generated and/or determined in step 2010. In step 2015, an in vivo fetal oximetry model may be augmented and/or personalized to the pregnant mammal and/or fetus using the physiological indicator received in step 2005 and, when step 2010 is executed, the calibration formula determined in step 2010.

In step 2020, light transmission data may be input into the augmented and/or personalized in vivo fetal oximetry model and an output may be received as a result (step 2025). The output may include an indication of an oximetry value for the fetus, such as a level of fetal hemoglobin oxygen saturation and a level of fetal tissue oxygen saturation. Optionally, the output from the augmented fetal in vivo fetal oximetry model may be evaluated and/or analyzed. For example, when the output received from the augmented fetal in vivo fetal oximetry model is a fetal oximetry value, the fetal oximetry value may be evaluated and/or analyzed to determine an indication, or not, of fetal distress. Additionally, or alternatively, execution of step 2030 may include comparing an oximetry value for the fetus for the fetus to a threshold fetal oximetry value and/or a previously determined oximetry value for the fetus to determine, for example, whether the oximetry value for the fetus is changing over time and/or whether the oximetry value for the fetus is too high or too low in comparison to the threshold. The output of step 2025 and/or a result of the evaluation/analysis of step 2030 may then be communicated to a display device like display device 155 (step 2035).

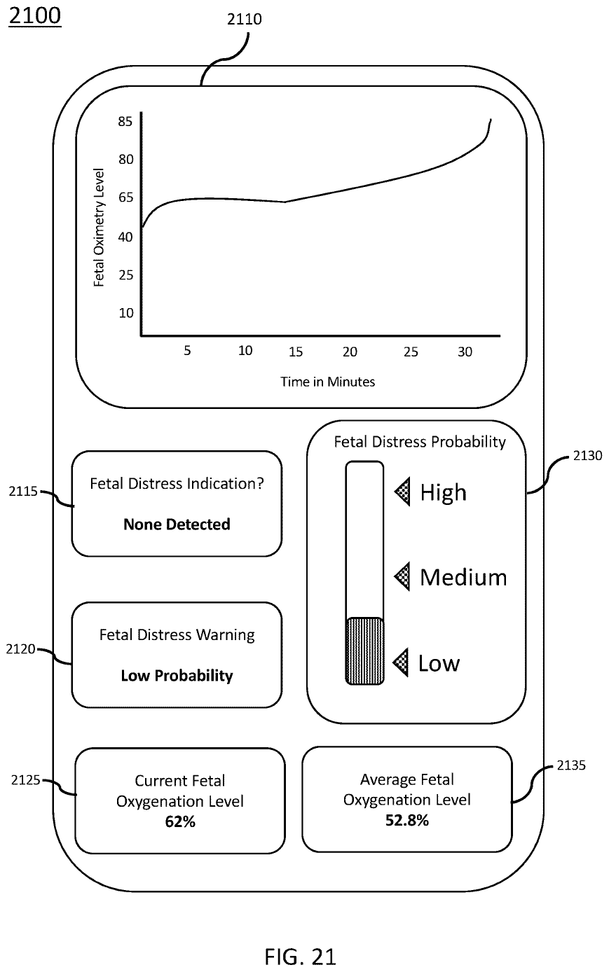

FIG. 21 is a screen shot of an exemplary user interface, or graphic user interface (GUI) 2100 that may be configured to display a result (e.g., a fetal oximetry value and/or indication of fetal distress) of executing one or more processes disclosed herein via, for example, one or more windows, icons, graphics, and/or text provided thereon. GUI 2100 may be displayed on, for example, display device 155, display device 14, and/or computer 13 responsively to instructions from, for example, computer 13 and/or cloud computing platform 11, and/or a component thereof (e.g., a processor, ASIC, and/or FPGA).

GUI 2100 includes a graph window 2110 that plots a plurality, or series of fetal oximetry level determinations, measurements, readings, and/or calculations taken over a period of time, in this instance 30 minutes. GUI 2100 further includes a fetal distress indication window 2115, a fetal distress warning window 2120, a current fetal oxygenation level window 2125, a fetal distress probability graphic window 2130, and an average fetal oxygenation level window 2135.

Fetal distress indication window 2115 may, for example, provide one or more messages regarding whether or not an indication of fetal distress has been detected such as “none detected” (as shown in FIG. 21). Other exemplary messages for fetal distress indication window 2115 include “potential distress detected” and “distress detected.” Additionally, or alternatively, fetal distress indication window 2115 may provide a message indicating an error condition and/or that additional information, or measurements, may be needed to assess fetal distress. Fetal distress warning window 2120 may, for example, an indication of a probability that the fetus is in distress such as “low probability” (as shown in FIG. 21). Other exemplary messages fetal distress warning window 2120 include, but are not limited to, a message indicating a probability level via words (e.g., high, medium, low) and/or numbers (e.g., 1-3; 1-5; 1-10; 1-100, etc.). Current fetal oxygenation level window 2125 may numerically display a value (in this case 62%) indicating a fetal oximetry level on any appropriate scale ((e.g., 1-3; 1-5; 1-10; 1-100, etc.)

Fetal distress, probability graphic window 2130 may graphically display a level of probably fetal distress as, for example, a bar graph (as shown in FIG. 21), a pie chart, a graph, and/or as a set of differently colored light that are coded to represent different levels of fetal distress probability (e.g., red indicate high distress probability and green indicates low distress probability). Average fetal oxygenation level window 2135 may display an average and/or a time-weighted average fetal oximetry value and/or level (in this case 52.8%) that may indicate an average fetal oximetry value over an interval of time (e.g., 5, 10, 15, 30, and/or 60 minutes).

Although FIG. 21 is in black and white, this need not always be the case as one or more windows of interface 2100 may provide information in the form of colors (e.g., red indicate high distress probability and green indicates low distress probability) that, in some cases, may indicate a, for example, a change in the fetus' oximetry level and/or a change in the fetus' distress probability. Additionally, or alternatively, a display device providing GUI 2100 may provide audible alerts and/or messages indicating, for example, a change in the fetus' oximetry level, a change in the fetus' distress probability, and the like.

It will be understood by those of skill in the art that GUI 2100 may not, in all circumstances, include each and every window shown in FIG. 21. In some embodiments, a GUI configured to display a result (of executing one or more processes disclosed herein may only include graph window 2110 and fetal distress, probability graphic window 2130. Alternatively, a GUI configured to display a result (of executing one or more processes disclosed herein may include fetal distress indication window 2115 and numerical value for a current fetal oxygenation level window 2125.

In some embodiments disclosed herein, one or more trained models, simulated fetal oximetry models, in vivo fetal oximetry models, calibration equations and/or correction factors may be used in combination to, for example, determine oximetry values for a patient, a fetus, and/or a pregnant mammal.

As described herein, in some embodiments, an in vivo fetal oximetry model may be personalized with one or more physiological indicators and this personalized in vivo fetal oximetry model may be referred to herein as an augmented in vivo fetal oximetry model. Additionally, or alternatively, an in vivo fetal oximetry model may be augmented (e.g., an augmented in vivo fetal oximetry model) with a formula that incorporates and/or corrects for a physiological indicator, such as skin color, that may remain relatively constant during a measurement session with a transabdominal fetal oximetry sensor. Additionally, or alternatively, an augmented in vivo fetal oximetry model may be personalized using one or more contemporaneously taken physiological indicators (e.g., maternal pulse oximetry information or respiratory rate) and may be updated on a periodic, as-needed, and/or continuous basis via, for example, input from one or more components of system 100.

We claim:

1. A method comprising:
receiving, by a processor, a physiological indicator for a pregnant mammal or the pregnant mammal's fetus;
receiving, by the processor, light transmission data, the light transmission data corresponding to an optical signal that is detected by a photodetector and converted into the light transmission data, the optical signal being a composite of light that passes through the pregnant mammal's abdomen and a fetus disposed within the pregnant mammal's abdomen;
inputting, by the processor, the light transmission data into an augmented fetal in vivo fetal oximetry model, the augmented fetal in vivo fetal oximetry model incorporating the physiological indicator; and
receiving, by the processor, an output from the augmented fetal in vivo fetal oximetry model, the output including an indication of an oximetry value for the fetus.

2. The method of claim 1, wherein the physiological indicator for the pregnant mammal is at least one of a hemoglobin oxygen saturation level, a tissue oxygen saturation level, a skin tone, a heartrate, a blood pressure, photoplethysmogram (PPG) signals or values, pulse, respiratory rate, uterine contraction information, duration of labor and delivery of a fetus, gestational age of the fetus, chronological age of the pregnant mammal, a hemoglobin oxygen saturation level, and a tissue oxygenation level.

3. The method of claim 1 or 2, wherein the oximetry value is at least one of a level of fetal hemoglobin oxygen saturation and a level of fetal tissue oxygen saturation.

4. The method of any of claims 1-3, further comprising:
determining, by the processor, a calibration formula for the pregnant mammal responsively to the physiological indicator, wherein the calibration formula is incorporated into the augmented fetal in vivo fetal oximetry model.

5. The method of any of claims 1-4, further comprising:
determining, by the processor, an indication of fetal distress using the oximetry value for the fetus; and
providing, by the processor, the indication of fetal distress to a display device.

6. The method of any of claims 1-5, further comprising:
comparing, by the processor, the oximetry value for the fetus to a threshold fetal oximetry value; and
providing, by the processor, an indication of the comparison to a display device.

7. The method of any of claims 1-6, further comprising:
querying, by the processor, a database of calibration equations for a calibration equation correlated to the physiological indicator;
receiving, by the processor, a calibration equation from the database responsively to the query, wherein the augmented fetal in vivo fetal oximetry model is personalized to the pregnant mammal or fetus using the received physiological indicator model.

8. The method of any of claims 1-7, further comprising:
querying, by the processor, a database of physiological indicator model for a physiological indicator model correlated to the physiological indicator;
receiving, by the processor, a physiological indicator model from the database responsively to the query, wherein the augmented fetal in vivo fetal oximetry model is personalized to the pregnant mammal or fetus using the received physiological indicator model.

9. The method of any of claims 1-8, further comprising:
receiving, by the processor, a plurality of physiological indicators over time; and
evaluating, by the processor, the plurality of physiological indicators, wherein the augmented fetal in vivo fetal oximetry model is personalized to the pregnant mammal using an evaluation of the received plurality of physiological indicators.

10. The method of claim 9, wherein each of the plurality of physiological indicators are a measurement of a physiological indicator for the pregnant mammal or fetus over time and the evaluation of the plurality of physiological indicators is a trend analysis.

11. A method comprising:
determining information regarding a blood oxygen value of a fetus, the determining comprising:
obtaining at least one signal indicating light detected from a pregnant mammal's abdomen and/or a fetus disposed in the pregnant mammal's abdomen following application of light to the pregnant mammal's abdomen; and
analyzing the at least one signal using a trained model personalized to the pregnant mammal to determine blood oxygen information used to determine the information regarding a blood oxygen value of the fetus, the trained model being personalized to the pregnant mammal using a physiological indicator for the pregnant mammal.

12. The method of claim 11, wherein the trained model is further personalized to the pregnant mammal using at least one of an optical and a geometric characteristic of the pregnant mammal's abdomen.

13. The method of claim 11 or 12, wherein the trained model is further personalized to the pregnant mammal using at least one of an optical and a geometric characteristic of the fetus.

14. The method of any of claims 11-13, wherein the trained model is further personalized to the pregnant mammal using a characteristic of the at least one signal.

15. The method of any of claims 11-14, wherein the trained model is further personalized to the pregnant mammal using at least one of a light scattering coefficient and a light absorption coefficient specific to the pregnant mammal.

16. The method of any of claims 11-15, wherein the trained model is personalized to the pregnant mammal using a skin color of the pregnant mammal's abdomen.

17. The method of any of claims 11-16, wherein the trained model is personalized to the pregnant mammal using an analysis of an image of the pregnant mammal's abdomen.

18. The method of any of claims 11-17, wherein the physiological indicator for the pregnant mammal is at least one of a hemoglobin oxygen saturation level, a tissue oxygen saturation level, a skin tone, a heartrate, a blood pressure, photoplethysmogram (PPG) signals or values, pulse, respiratory rate, uterine contraction information, duration of labor and delivery of a fetus, gestational age of the fetus, chronological age of the pregnant mammal, a hemoglobin oxygen saturation level, and a tissue oxygenation level.

19. The method of any of claims 11-18, wherein the trained model is personalized to the pregnant mammal using a calibration formula that is responsive to one or more characteristics of the pregnant mammal.

20. The method of any of claims 11-19, wherein the information regarding the blood oxygen value of the fetus is a level of fetal hemoglobin oxygen saturation.

21. The method of any of claims 11-20, wherein the information regarding the blood oxygen value of the fetus is a level of fetal tissue oxygen saturation.

22. The method of any of claims 11-21, further comprising:
determining whether the fetus has fetal hypoxia or fetal hypoxemia using the blood oxygen value; and
providing an indication of a determination that the fetus has fetal hypoxia or fetal hypoxemia to a display device.

23. A method comprising:
determining information regarding a blood oxygen value of a patient, the determining comprising:
obtaining a physiological indicator for the patient and at least one signal indicating light detected from the patient following application of light to the patient; and
analyzing the at least one signal using at least one trained model and the physiological indicator of the patient to determine blood oxygen information for the patient.

24. The method of claim 23, wherein the trained model is personalized to the patient using the physiological indicator.

25. The method of claim 23 or 24, wherein the physiological indicator is a measurement or a characteristic of the patient.

26. The method of any of claims 23-25, wherein the trained model is personalized to the patient using at least one of a skin tone of the patient and skin melanin content for the patient.

27. The method of any of claims 23-26, wherein the trained model is personalized to the patient using a characteristic of the at least one signal.

28. The method of any of claims 23-27, wherein the trained model is personalized to the patient using at least one of a light scattering coefficient and a light absorption coefficient specific to the patient.

29. The method of any of claims 23-28, wherein the information regarding the blood oxygen value of the patient is a level of hemoglobin oxygen saturation for the patient.

30. The method of any of claims 23-29, wherein the information regarding the blood oxygen value of the patient is a level of tissue oxygen saturation for the patient.

31. The method of any of claims 23-30, further comprising:
determining whether the patient has hypoxia or hypoxemia using the blood oxygen value; and
providing an indication of a determination that the patient has hypoxia or hypoxemia to a display device.

32. A computer-implemented method comprising:
receiving physiological indicator regarding a pregnant mammal or a fetus disposed within the pregnant mammal's abdomen; and
generating a physiological indicator model for the pregnant mammal or fetus responsively to the received physiological indicator.

33. The computer-implemented method of claim 32, further comprising:
receiving light transmission data, the light transmission data corresponding to an optical signal that is incident on the pregnant mammal's abdomen and the fetus disposed within the pregnant mammal's abdomen and is detected by a photodetector and converted into the light transmission data; and
determining an oximetry value for the fetus by applying the physiological indicator model to the light transmission data.

34. The computer-implemented method of claim 32 or 33, wherein the light transmission data is received from at least one of a frequency domain near infrared spectroscopy system, a continuous wave near infrared spectroscopy system, and a time of flight near infrared spectroscopy system.

35. The computer-implemented method of any of claims 32-34 wherein the optical signal is a first optical signal, the photodetector is a first photodetector of a first spectroscopy system and the light transmission data is a first set of light transmission data, the method further comprising:
receiving a second set of light transmission data, the second set of light transmission data corresponding a second optical signal that is incident on the pregnant mammal's abdomen and a fetus disposed within the pregnant mammal's abdomen and is detected by a photodetector of a second spectroscopy system and converted into the second set of light transmission data; and
determining an oximetry value for the fetus by applying the calibration equation to the second set of light transmission data.

36. The computer-implemented method of claim 35, wherein the second spectroscopy system is a continuous wave near infrared spectroscopy system.

37. The computer-implemented method of claim 34, 35, or 36, wherein the physiological indicator for the pregnant mammal includes an oxygen saturation level for the pregnant mammal.

38. The computer-implemented method of claim 37, wherein the maternal oxygen saturation level is received from a pulse oximeter.

39. A system comprising:
a processor in communication with a memory; and
the memory, the memory being configured to store a set of instructions thereon, which when executed by the processor cause the processor to execute any of the methods of claims 1-10.

40. A system comprising:
a processor in communication with a memory; and
the memory, the memory being configured to store a set of instructions thereon, which when executed by the processor cause the processor to execute any of the methods of claims 11-22.

41. A system comprising:
a processor in communication with a memory; and
the memory, the memory being configured to store a set of instructions thereon, which when executed by the processor cause the processor to execute any of the methods of claims 23-31.

42. A system comprising:
a processor in communication with a memory; and
the memory, the memory being configured to store a set of instructions thereon, which when executed by the processor cause the processor to execute any of the methods of claims 32-38.
